(19) United States
(12) Patent Application Publication
(10) Pub. No.: US 2026/0211915 A1
(43) Pub. Date: Jul. 23, 2026

(54) INTEGRATING PLATFORMS FOR AN ARTIFICIAL INTELLIGENCE BASED DIGITAL COMPANION

(71) Applicant: Roy Schoenberg, Boston, MA (US)

(72) Inventor: Roy Schoenberg, Boston, MA (US)

(21) Appl. No.: 19/438,069

(22) Filed: Dec. 31, 2025

Related U.S. Application Data
(60) Provisional application No. 63/746,615, filed on Jan. 17, 2025.
(60) Provisional application No. 63/803,022, filed on May 9, 2025.

(51) Int. Cl. G06F 16/3329; G08B 21/04; H04L 67/306

(52) U.S. Cl. CPC G06F 16/33295; G08B 21/04; H04L 67/306

(57) ABSTRACT

Personalized communication systems for elderly users include an artificial intelligence (AI)-based digital companion for a user. The user is onboarded based on answers to a set of questions. A profile for the user is generated. Recent data associated with the user is accessed, from a hardware storage device. An agenda including topics to be addressed with the user is generated. A prompt, based on the topic for the interaction with the user and a context of the topic determined from the generated profile and the recent data is transmitted to an AI engine trained to generate a conversational output relevant to the user. Output data specifying one or more instructions for interacting with the user is received from the AI engine. A communication with the user is established, through a communication channel with a user device of the user, in accordance with the one or more instructions.

### CROSS-REFERENCE TO RELATED APPLICATIONS

This application claims the benefit of priority under 35 U.S.C. § 119 to U.S. Provisional Patent Application No. 63/746,615, filed on Jan. 17, 2025, and U.S. Provisional Patent Application No. 63/803,022, filed on May 9, 2025. The entire contents are incorporated by reference herein.

### TECHNICAL FIELD

The present disclosure relates to data transmission networks. More particularly, implementations of the present disclosure are directed to an artificial intelligence (AI) based digital companion for transmitting customized, digital communications.

### BACKGROUND

The unprecedented growth of the aging population worldwide is driven by increased life expectancy and declining birth rates. A pressing concern associated with this trend is the risk of social isolation among the elderly, as well as declining self-sufficiency and fragility with age and solitude. Solitude (e.g., isolation) is not merely a social issue; it has profound implications for physical and mental health. Factors contributing to isolation include mobility limitations, loss of family or friends, technological barriers, and inadequate community support systems.

### SUMMARY

Described herein is an AI-based digital companion engine for simulating a familiar relationship with an older adult (e.g., an elder). The AI-based digital companion engine described herein simulates such a familiar relationship by conversing with the older adult, but also by being watchful and vigilant. For example, while having regular conversations with the older adult, the AI-based digital companion engine tracks various parameters (e.g. sentiment, confusion, anxiety and even if the fridge door was opened this morning or if a motion sensor indicated the older adult is still in bed) and uses this analysis to both influence its conversation with the older adult (e.g., by asking “why didn't you have breakfast this morning?”) or to update/escalate to human caregivers. This includes both paid caregivers (e.g., home care agencies or senior living community staff members) as well as unpaid caregivers (e.g., family members and adult children).

Simulating such a familiar relationship with an older adult poses challenges, many of which are specific to the older adult population.

First, there is often no data to learn from. There are often no books or documents for AI to ingest to machine learn about the elder. AI cannot create intimacy and familiarity if it cannot ingest data that creates the intimacy. To solve this problem, the AI-based digital companion engine will autonomously call and interview those (e.g., family, friends, and so forth) who know the elder. The AI-based digital companion engine initiates these communications and uses this information to create a profile for the elder, as described herein.

Second, AI is reactive. AI is designed to respond to prompts, but it is unlikely that elders will initiate interactions with AI. That is, if a companion engine relies on seniors to initiate an interaction, this model will likely fail. To address this problem, the AI-based digital companion engine described herein will initiate the communication. The AI-based digital companion engine will trigger an AI model and start a communication with the elder. That is, the AI-based digital companion engine itself is doing the prompting of the AI model, rather than relying on an elder to do the prompting.

Third, AI builds on documents of the past. Unlike search engines, AI knowledge is not current. It is not aware of what is happening today or tomorrow, which is what keeps daily conversations relevant. To address this, the AI-based digital companion engine includes skills or non-AI capabilities for accessing content and information. That is, the AI-based digital companion engine includes search-like insights into the otherwise AI-operated conversation. Generally, a skill includes non-AI-based content (or information) or a content/information provider (e.g., a website that provides the news or daily jokes, a social media platform, a sensor on a smart refrigerator that provides status updates regarding usage, and so forth). That is, skills are designed to reach out to the outside world and incorporate information from the outside world into the communication with the elder. For example, the AI-based digital companion engine uses the skills functionality to receive information from the sensor on a smart refrigerator, with the received information specifying that the refrigerator has not been opened all morning. Based on that information, the AI-based digital companion engine determines that perhaps the elder has not gotten out of bed this morning, since the refrigerator has not been opened. Based on this insight, the AI-based digital companion engine initiates a conversation with the elder and asks the elder how he or she is feeling today. Based on the response (or a lack of response), the AI-based digital companion engine may generate an escalation notification, e.g., to notify family members, as described herein.

Fourth, AI is accessed via technology. As such, accessing AI may be difficult for some elders, e.g., due to issues of lack of digital literacy. The AI-based digital companion engine described herein addresses this problem by simply using the phone to initiate a conversation. An elder does not need to learn AI or new technology. The elder simply needs to use what he or she already knows—picking up a telephone call.

The AI-based digital companion engine described herein can be used in various manners. For example, the AI-based digital companion engine can be used for staffing efficiency, e.g., serving alongside caregivers and spacing caregiver visits. The AI-based digital companion engine can also be the primary presence, e.g., escalating to caregivers as backup and as needed. Additionally, the AI-based digital companion engine may be an ambient feature of a senior living unit.

In an implementation, the system described herein includes a companion engine to establish companionship (and not authority) with a user, such as an older adult. Generally, a companion engine includes, e.g., an AI engine that simulates a familiar personal companion that is an indispensable partner in the daily reality of a senior. This provides a foundation onto which health care messages can be added. The system described herein uses AI to continuously learn a person, not a process. The companion engine is configured for a unilateral, proactive relationship, in which the companion engine calls, check-ups on and talks—a lot—with the senior. This provides for a flowing, interesting conversation, with healthcare woven in. The companion engine can also watch over the senior, detect patterns and events, escalate to humans (e.g., trigger notifications), as needed. The result of this is a tangible benefit of extending the time-horizon of self-sufficiency.

In an implementation, a computer-implemented method includes: simulating, by a computer system, an artificial intelligence (AI)-based digital companion for a user, including: onboarding the user by receiving, from a user device, one or more answers to one or more interview questions, based on the one or more received answers, generating a profile for the user, retrieving, from one or more external data sources by one or more application program interfaces (APIs), recent data associated with the user, with the recent data having occurred within a threshold amount of time, accessing, from a hardware storage device, an agenda for the user, the agenda including a plurality of topics to be addressed with the user, based on the generated profile, the recent data and the agenda, selecting a topic for an interaction with the user, generating a prompt, based on the topic for the interaction with the user and a context of the topic determined from the generated profile and the recent data, transmitting the prompt to an AI engine trained to generate a conversational output relevant to the user, receiving, through an API of the AI engine, output data specifying one or more instructions for interacting with the user, and establishing, through a communication channel with a user device of the user, a communication with the user in accordance with the one or more instructions.

In an aspect, combinable with the previous aspect, the topic is a first topic and wherein the method further includes: receiving, through the communication channel, input data from the user device of the user, generating of transcript of the communication based on the received input data, identifying, based on the generated transcript, one or more attributes of the user, responsive to the identifying, updating a profile of the user based on the one or more attributes, and transmitting a second topic to the AI engine, with the second topic specifying a request for another interaction with the user and further specifying the updated profile. In another aspect, combinable with any of the previous aspects, the recent data associated with the user is retrieved using a digital identifier by accessing a computing system associated with the digital identifier, the computing system hosting the one or more data records through an application interface that is different from another application interface associated with one or more other data records associated with a different computing system associated with another digital identifier. In another aspect, combinable with any of the previous aspects, retrieving the recent data associated with the user associated with the digital identifier includes accessing authorization data associated with the digital identifier, and accessing, using the authorization data, a computing system hosting the one or more data records. In another aspect, combinable with any of the previous aspects, retrieving the recent data associated with the user associated with the digital identifier includes activating a sensor for initiating data collection in real time. In another aspect, combinable with any of the previous aspects, the recent data associated with the user is constrained to a threshold time period defining topic currency. In another aspect, combinable with any of the previous aspects, the computer-implemented method includes activating a device for generating an alert associated with the one or more instructions. In another aspect, combinable with any of the previous aspects, the device includes a medical assistance device and the alert associated with the one or more instructions corresponds to an event task. In another aspect, combinable with any of the previous aspects, the computer-implemented method includes determining a context for the topic, wherein the context defines behavioral and user data associated to the topic. In another aspect, combinable with any of the previous aspects, the computer-implemented method includes establishing, through an additional communication channel a communication between the user device of the user and a provider gateway system for rendering a visual representation of a service provider within a graphical user interface of the user device.

The described subject matter can be implemented using a computer-implemented method; a non-transitory, computer-readable medium storing computer-readable instructions to perform the computer-implemented method; and/or a computer-implemented system comprising one or more computer memory devices interoperably coupled with one or more computers and having tangible, non-transitory, machine-readable media storing instructions that, when executed by the one or more computers, perform the computer-implemented method/the computer-readable instructions stored on the non-transitory, computer-readable medium.

The details of one or more implementations of the subject matter of this specification are set forth in the Detailed Description, the Claims, and the accompanying drawings. Other features, aspects, and advantages of the subject matter will become apparent to those of ordinary skill in the art from the Detailed Description, the Claims, and the accompanying drawings.

### DESCRIPTION OF DRAWINGS

FIG. 1A is a block diagram illustrating an example system for an artificial intelligence based digital companion, according to an implementation of the present disclosure.

FIG. 1B is a block diagram illustrating another example system for an artificial intelligence based digital companion, according to an implementation of the present disclosure.

FIG. 1C is a block diagram illustrating another example system for an artificial intelligence based digital companion, according to an implementation of the present disclosure.

FIG. 2A shows an example system for onboarding an artificial intelligence based digital companion, according to an implementation of the present disclosure.

FIG. 2B shows an example system for managing skills of an artificial intelligence based digital companion, according to an implementation of the present disclosure.

FIG. 2C shows an example data flow for processing data for connecting user devices with companion engines for personalized communication sessions, according to an implementation of the present disclosure.

FIG. 2D illustrates that artificial intelligence-based digital companion engine is configured to communicate with seniors using various modalities, according to an implementation of the present disclosure.

FIG. 3 is a block diagram illustrating an example data ranking, according to an implementation of the present disclosure.

FIG. 4 is a block diagram illustrating an example agenda dispatcher, according to an implementation of the present disclosure.

FIG. 5 is a block diagram illustrating an example data retrieval system, according to an implementation of the present disclosure.

FIG. 6 shows an example data flow for generating personalized interactions, according to an implementation of the present disclosure.

FIG. 7 is a block diagram illustrating an example system core architecture for executing a companion engine for personalized communication sessions, according to an implementation of the present disclosure.

FIG. 8 is a flowchart illustrating an example of a computer-implemented method for executing a companion engine for personalized communication sessions, according to an implementation of the present disclosure.

FIG. 9 is a block diagram illustrating an example of a computer-implemented system used to provide computational functionalities associated with described algorithms, methods, functions, processes, flows, and procedures, according to an implementation of the present disclosure.

Like reference numbers and designations in the various drawings indicate like elements.

### DETAILED DESCRIPTION

AI models face significant challenges when integrated into personalized communication systems for elderly users. A primary issue is the absence of standardized, domain-specific datasets for training the AI model, which makes it difficult to achieve nuanced personalization without extensive manual data curation. Furthermore, due to their inherently reactive architecture, most AI models struggle to generate or ingest contextual signals that foster emotional intimacy, relying instead on transactional exchanges rather than proactive engagement. Another limitation lies in their dependence on historical conversational data, which often results in outdated or contextually irrelevant responses, reducing the perceived authenticity and freshness of interactions. Additionally, these models are typically accessed through intermediary applications or complex user interfaces, creating usability barriers for older adults who may have limited technical literacy. This combination of data scarcity, reactive design, temporal staleness, and accessibility challenges underscores the need for adaptive AI frameworks capable of dynamic learning, multimodal context integration, and simplified interaction layers tailored to the cognitive and emotional needs of the aging population.

The following detailed description describes an AI-based digital companion engine for providing personalized communication sessions with a user, the AI-based digital companion engine being configured to keep conversation lively, familial and current. A personal companion is simulated by a computer system (e.g., executing the AI-based digital companion engine), which begins by onboarding a user (elder) during user-friendly prescheduled calls. The onboarding sessions are configured for building a personalized dataset, by receiving, from a user device associated with the user, one or more answers to interview questions. The answers to these questions are often received by the companion engine autonomously calling and interviewing those who know the elder. A profile for the elder is generated based on the received answers. Recent data associated with the user is retrieved from one or more external data sources via application program interfaces (APIs), with the data constrained to a threshold time period for maintaining topic currency. An agenda for the elder is accessed from a hardware storage device, the agenda comprising a plurality of topics to be addressed. A topic for interaction with the user is selected based on the generated profile, the recent data, and the agenda. A prompt for the interaction is generated using the selected topic and its context determined from the profile and recent data. The prompt is transmitted to an AI engine trained to proactively generate conversational output relevant to the elder. Output data is received from the AI engine through its API, specifying one or more instructions for interacting with the elder. Communication with the elder is established through a channel connected to the user device, mimicking a regular phone call or another user-friendly communication method preferred by the user to minimize technological anxiety. In some examples, the companion engine initiates a telephonic communication with the elder or a text message communication.

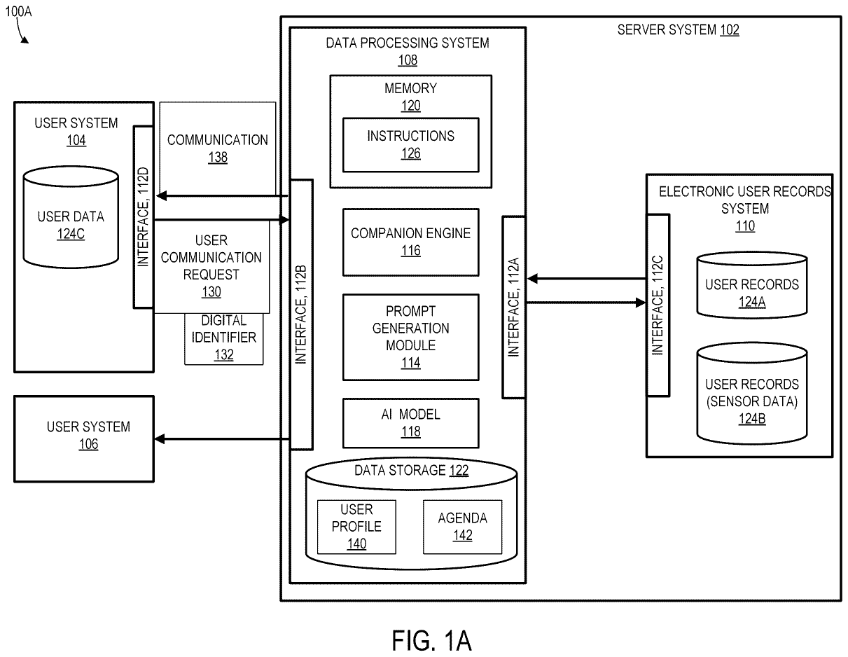

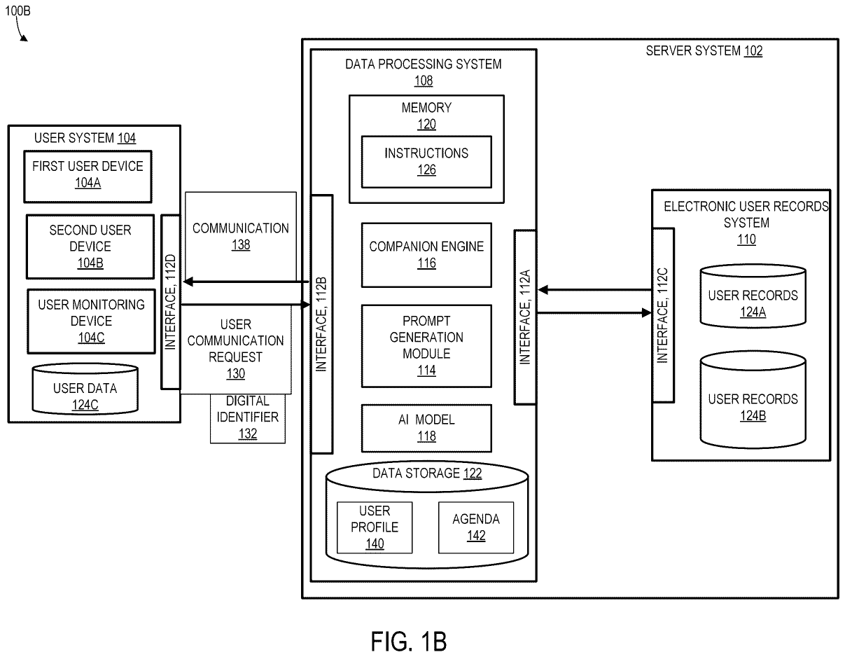

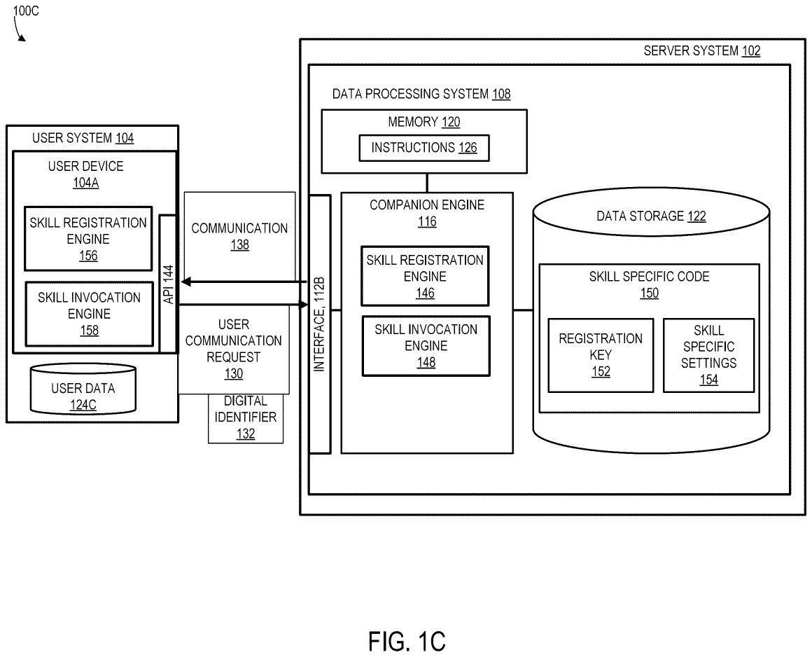

FIG. 1A is a block diagram illustrating an example system 100A for an artificial intelligence based digital companion, according to an implementation of the present disclosure. The example system 100A includes a server system 102, a user system 104, and a user system 106. The server system 102 is configured to establish communication channels for connecting the user system 104 with an AI-based digital companion engine 116. The server system 102 includes a data processing system 108 and an electronic user records system 110. The data processing system 108 includes interfaces 112A, 112B, a prompt generation module 114, the AI-based digital companion engine 116, an AI model 118, a memory 120, and data storage 122.

The data processing system 108 is configured to communicate with the electronic user records system 110, to retrieve and process user records 124A, 124B for generating personalized communication with the user system 104 and for routing the communication 138 to the respective user system 104 according to a set of instructions 126. For example, the data processing system 108 is configured to simulate a personal companion for the user system 104 operated by an elder by executing a series of coordinated functions. The data processing system 108 uses the interfaces 112A and 112B for receiving, from the user system 104 associated with the elder, one or more answers to interview questions during onboarding. In another example, the data processing system 108 receives, from the user system 106 (e.g., associated with a family member or friend of the elder), one or more answers to interview questions about the elder during onboarding. Based on the answers, the data processing system 108 utilizes the memory 120 and data storage 122 to generate a personalized profile 140 for the user (elder). The data processing system 108 retrieves recent data 124A, 124B associated with the user from one or more external data sources, including the electronic user records system 110. As described above, this recent data 124A, 124B may be sensor data collected from a sensor of the elder's smart refrigerator, with the sensor data specifying when was the last time the refrigerator was used. The data processing system 108 can accesses an agenda 142 stored in the data storage 122. The agenda 142 can include multiple topics to be addressed during the personalized communication with the user system 104. Not all of the topics are AI-based. Some of the topics may be to simply interact with the elder, e.g., tell a joke or check-up on the elder if he/she has not yet gotten out of bed (e.g., as indicated by the sensor data 124B). Other topics are AI-based. For these topics, using the prompt generation module 114, the data processing system 108 selects a topic from the agenda 142 for interaction based on the generated profile 140, recent data, and generates a context-aware prompt. The prompt is transmitted to the AI model 118, which is trained to produce conversational outputs (e.g., aligned with user's goals, that are relevant to the user, aligned with the selected agenda topic and so forth), which are transmitted to the user system 104 to establish a personalized communication with the user system 104. The companion engine 116 receives output data from the AI model 118 through an API, specifying instructions for interacting with the elder. Finally, the data processing system 108 establishes a secure communication channel with the user system 104 to deliver the interaction (e.g., as communication 138) in accordance with the received instructions 126. The data processing system 108 can adjust the personalized communication 138 with the user system 104 based on user records 124A, 124B and user data 124C. For example, the data processing system 108 uses the interface 112A to retrieve user data 124C from a user system 104. The data processing system 108 can retrieve the user data 124C in response to receiving a user communication request 130 through the interface 112B (e.g., a provider gateway system) that can be communicatively coupled to the interface 112D of the user system 104. The user communication request 130 can include a digital identifier 132 of a user providing the user input by interacting with the user system 104.

FIG. 1B is a block diagram illustrating another example system for an artificial intelligence based digital companion, according to an implementation of the present disclosure. The example system 100B shown in FIG. 1B can include or be coupled to the example system 100A described with reference to FIG. 1A. The example system 100B includes a server system 102 and a user system 104. The server system 102 includes a data processing system 108 and an electronic user record system 110. The data processing system 108 includes interfaces 112A, 112B, a prompt generation module 114, a companion engine 116, an AI model 118, a memory 120, and data storage 122.

The user system 104 includes a first user communication device 104A, a second user device 104B, and a user monitoring device 104C. The first and second user communication devices 104A, 104B can include any type of computing device configured to facilitate secure and real-time bidirectional communication with the server system 102. For example, the user communication device 104A can be implemented as a smartphone, tablet, or other computing device equipped with a microphone a speaker, a camera, a user interface, a processor, memory, and a network interface supporting wired or wireless connectivity such as Wi-Fi, LTE, or 5G. The first and second user communication devices 104A, 104B can include an interface 112D capable of handling bidirectional text messaging, audio communications, and audio-video data streams using protocols such as Web Real-Time Communication (WebRTC) or Real-Time Transport Protocol (RTP) for low-latency transmission, in addition to secure transmission of user data 124C via HTTPS or TLS-encrypted channels. To ensure compliance with privacy standards, the user communication device 104A can also support authentication mechanisms such as OAuth or token-based access control for API interactions, and can incorporate hardware components like cameras, microphones, and biometric sensors to enable identity verification and support bidirectional communication sessions. The first user communication device 104A can be designated as preferred for communications with the companion engine using the skill registration engine 142 and the skill invocation engine 140, shown in FIG. 1C. The second user communication device 104B can be a backup device that can be activated if the first user communication device 104A is unavailable.

The user monitoring device 104C can include a sensor to non-invasively or minimally invasively collect one or more user data. The user data can include bio signals such as heart rate, blood pressure, electrocardiogram, glucose level, temperature, blood oxygenation, and other data indicative of a user condition. In addition, the user assistance (e.g., medical) device 104B can incorporate wearable technologies, such as smart health monitors or biosensors, equipped with embedded processors, wireless communication modules, and secure data transmission protocols such as TLS or DTLS. user health monitoring device 104C can collect user data 124C that can be transmitted to the server system 102 to support real-time telemetry and bidirectional communication with the data processing system 108 through standardized healthcare protocols enabling continuous monitoring and adaptive adjustments. In some implementations, each of the user communication device 104A, the second user device 104B, and the user health monitoring device 104C includes hardware components such as integrated sensors for vital signs, battery management systems for uninterrupted operation, and encrypted storage for temporary data buffering, ensuring compliance with privacy security standards.

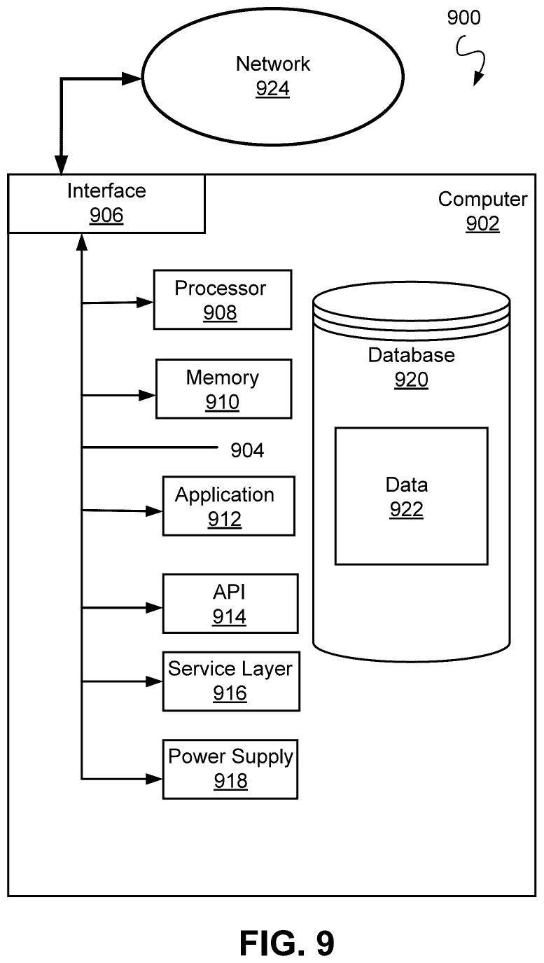

FIG. 1C is a block diagram illustrating another example system 100C for an artificial intelligence based digital companion, according to an implementation of the present disclosure. As shown in FIG. 1C, the example system 100C includes server system 102 and a user system 104, connected via the interface 128 and an API 144 (similar to API 914, described with reference to FIG. 9).

The server system 102 includes a data processing system 108 and a data storage 122. The data processing system 108 includes a memory 120, which stores instructions 126 for executing processes, and a companion engine 116 that mirrors client-side functionality. The companion engine 116 engine includes a skill registration engine 146 and a skill invocation engine 148, enabling the server system 102 to handle skill registration and invocation tasks for configuring personalized communication sessions. The skill registration engine 146 can facilitate an authorized skill service provider (e.g., user system 106 in FIG. 1) to register skills into the data storage 122. The registered skills can be available to be incorporated in scheduled personalized communications with the user devices 104A.

The skill registration engine 146 can be responsible for multiple critical functions that enable seamless integration of skills within the user device 104A. The skill registration engine 146 performs authentication of the service provider to ensure secure communication and prevent unauthorized access. Once authenticated, the skill registration engine 146 establishes an internal identification for the specific skill, creating a unique mapping that allows the data processing system 108 to recognize and manage the skill efficiently. The skill registration engine 146 translates the service provider's configuration parameters into a format compatible with the user interface of the device, enabling clear and user-friendly display of settings for customization. After successful registration, the skill registration engine 146 confirms the process to user system 106 by transmitting a shared identifier, which serves as a reference for future interactions. Additionally, the engine monitors the registration process for errors and sends alerts if any issues occur, ensuring reliability and transparency in skill onboarding. The skill registration engine 146 can transmit to the user device a registration permission key 152 including an indicator of a registration success or error code, the registration success including a link to the skill directory.

The companion engine 116 can execute a series of post-registration operations once a skill has been successfully registered. The post-registration operations begin with exposing the registered skill in a centralized skill directory, making it discoverable for user interaction and system-level management. The companion engine 116 renders the skill's configurable parameters on the skill settings page within the directory, stored in the data storage 122, enabling users to customize behavior through an intuitive interface. The companion engine 116 captures and stores service provider-defined values for each setting 154 on a per-user account basis, ensuring personalized configurations. Furthermore, the companion engine 116 associates registered skills with scheduled, personalized scheduled communication sessions, which may include deterministic invocation logic to trigger skills at predefined times or under specific conditions. The companion engine 116 manages lifecycle controls by enabling or disabling a configured skill and toggling the invocation of an artificial intelligence model responsible for processing skill logic and generating communication outputs. To maintain operational integrity, the companion engine 116 performs periodic health checks to verify skill availability and automatically disables offline or non-responsive skills, thereby ensuring a consistent and reliable user experience.

In some implementations, the companion engine 116 can operate as an ambient, context-aware feature that remains in a low-power standby state until activated through user input, such as voice commands, gesture recognition, or touch-based triggers. Upon activation, the engine initiates an unscheduled personalized communication session without requiring prior scheduling. The communication session begins with an introductory phase, during which the companion engine 116 dynamically retrieves and prioritizes relevant conversational skills using the skill invocation engine 148. The invocation engine 148 employs a multi-step process that includes semantic intent parsing of the user's initial input, context matching against stored user profiles and historical conversation data, and real-time skill ranking based on relevance and priority. Skills may include news updates, health monitoring, or IoT device status alerts, which are fetched through API calls or event-driven microservices. The architecture leverages asynchronous task execution and low-latency communication protocols to ensure rapid skill deployment, while maintaining conversational continuity and personalization. This design enables the AI-based companion to deliver spontaneous, contextually rich interactions that adapt to user needs in real time, enhancing engagement and reducing reliance on rigid scheduling frameworks. The skill invocation engine 148 also enables the AI-based digital companion engine 116 to interact with a user in a non-AI based manner. For example, the agenda described herein may specify a given time for telling a joke or providing a news update (e.g., about news topics that are of interest to the elder). At the preset (triggered or scheduled) time, the skill invocation engine 148 can request from one or more external data sources (e.g., skill providers) specified information (e.g., a joke, news, and so forth). The skill invocation engine 148 can receive the requested information and transmit it (e.g., as communication 138) to the user system 104.

The skill invocation engine 148 is configured to orchestrate personalized communication sessions by preparing the necessary context before a scheduled call is initiated. For example, the skill invocation engine 148 dynamically sets up a communication workflow tailored to the user of the user device 104A, ensuring that relevant information and preferences are incorporated. To achieve this, the skill invocation engine 148 securely connects to the authorized skill service provider (e.g., user system 106) using authenticated channels, retrieving a subset of registered skills that are pertinent to the upcoming interaction. The skills may include personalized prompts, contextual data, or AI-driven enhancements. The skill invocation engine 148 aggregates and processes the information to generate a comprehensive main prompt, which serves as the foundation for the personalized communication experience. The prompt can include deterministic invocation logic, ensuring that specific skills are triggered at the right moment during the session. The skill invocation engine 148 can scrutinize fresh insights related to the user to be able to update ongoing and subsequent personalized communication sessions and removes skills at particular times to avoid repetitive recursions of topics, to add variation of covered subjects during personalized communication sessions. Additionally, the skill invocation engine 148 manages error handling, fallback strategies, and latency optimization to guarantee a smooth and responsive user experience.

The data storage 122 serves as a persistent repository for skill-related assets and includes skill specific code 150, which is essential for enabling and managing individual skills. The skill specific code 150 includes a registration key 152, a secure token or cryptographic identifier used to validate and authorize skill registration requests, facilitating that only authenticated service providers can register skills within the system. In addition, the data storage 122 contains skill specific settings 154, which define the operational parameters and configuration details for each skill. The skill specific settings 154 can include user preferences, invocation rules, access permissions, and integration endpoints, allowing the system to tailor skill behavior to individual user accounts. By maintaining the registration key 152 and the skill specific settings 154 in a structured and secure manner, the data storage 122 ensures high availability, integrity, and scalability for skill execution across multiple sessions and devices.

The user system 104 includes a user device 104A, which includes a skill registration engine 156 and a skill invocation engine 158. The skill registration engine 156 can be configured to handle the complete lifecycle of skill registration by leveraging skill registration data provided through the device's user interface. The skill registration data includes critical elements such as the skill name, skill icon, skill description, skill server URL, skill-specific parameters, health check URL, and a registration permission key for authentication and authorization. Once the user inputs the skill registration details, the skill registration engine 156 validates the data and packages it into a structured registration payload. The payload is securely transmitted to the companion engine 116 on the server side via an API call. The companion engine subsequently stores the registration data in data storage 122, ensuring persistence and availability for future invocation. The skill registration engine includes error handling mechanisms to detect invalid parameters, missing keys, or connectivity issues, and provides real-time feedback to the user interface for corrective actions. The skill registration process ensures that skills are registered accurately, securely, and in compliance with system-level policies. In this example, the user system 104 includes the skill registration engine 156 and the skill invocation engine 158. In another example, the user system 106 (FIG. 1B) includes the skill registration engine 156 and the skill invocation engine 158, e.g., when a friend or family member selects or specifies the skills (e.g., content sources) to be used for an elder.

The skill invocation engine 158 manages the execution of the skills to generate a companion communication session. The companion communication session can be initiated in view of receiving a trigger to initiate the session. The companion communication session can be an audio/video personalized session including real-time responses and discussion statements generated according to the skill specific settings and user preferences, interests and updates. The user system includes user data 124C, which stores user-specific information relevant to skill operations. Communication between the user system 104 and the server system 102 occurs through the API 141, transmitting a user communication request 130 along with a digital identifier 132 for authentication or identification purposes to facilitate secure skill registrations and personalized communication sessions.

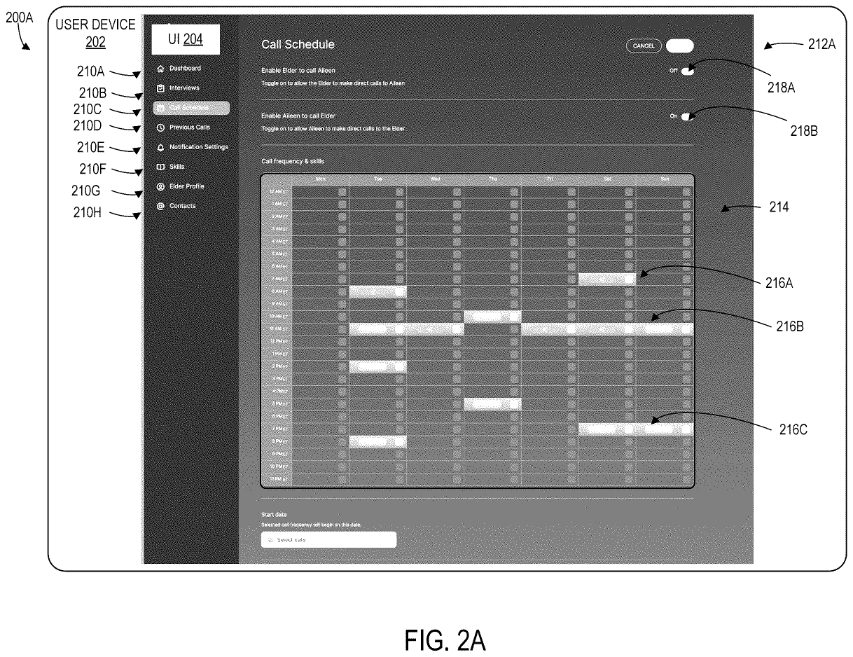

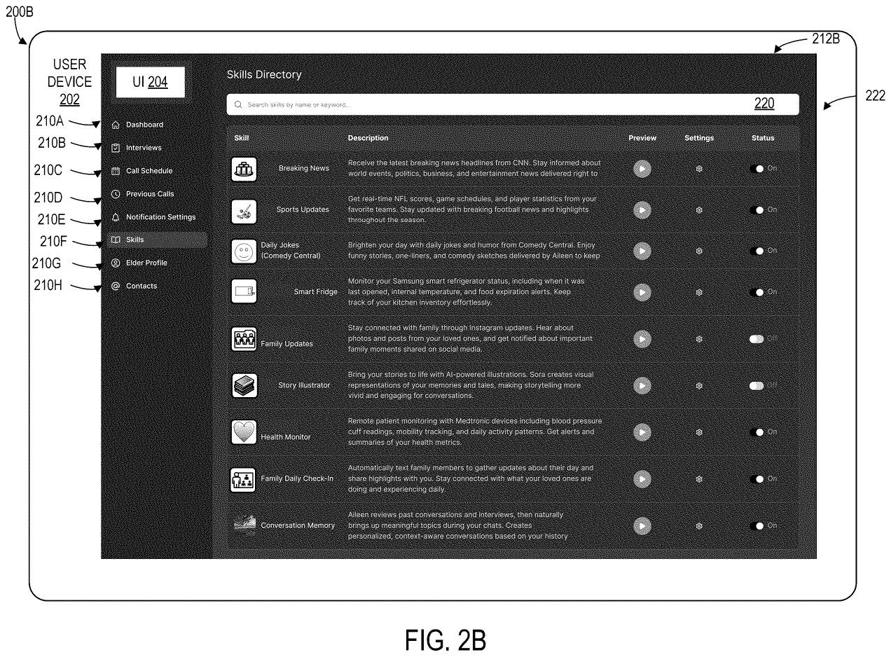

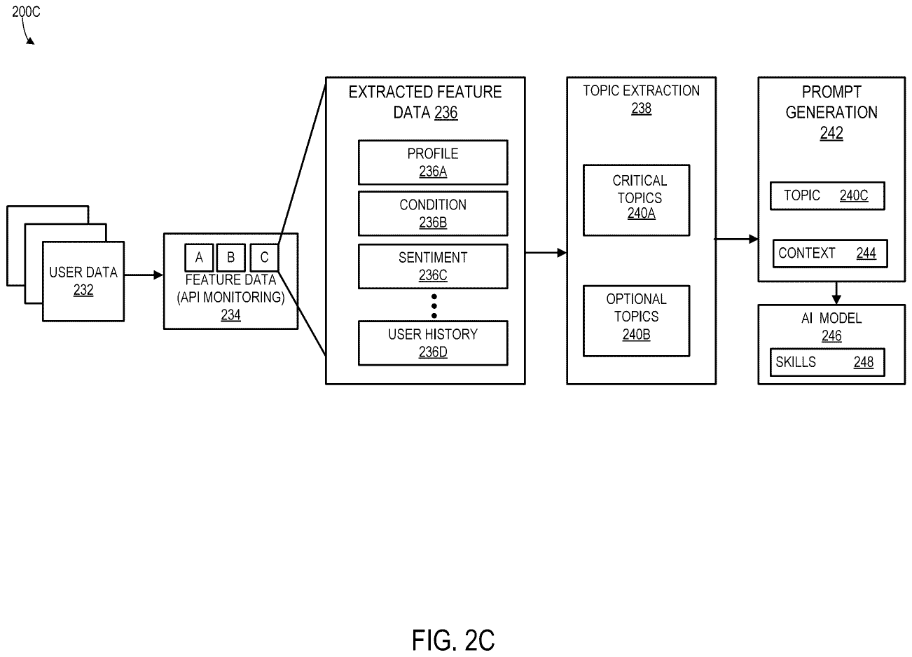

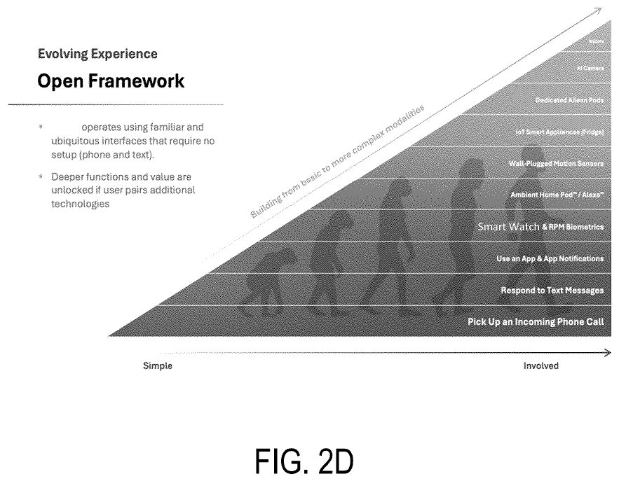

FIG. 2A shows an example system 200A for onboarding an artificial intelligence based digital companion, according to an implementation of the present disclosure. The example system 200A can include a user device 202 and a user interface 204A. In this example, the user device 202 may be used by a user that is distinct from the elder, e.g., to allow a family member “jumpstart” AI for the elder by specifying when the digital companion engine should initiate a communication with the elder. The scheduled time for the communication, sets a time when the digital companion engine triggers AI and then joins or otherwise brings the senior into the conversation.

The user interface 204A can include a quick access bar 206 and a schedule interface 208. The quick access bar 206 can include a dashboard icon 210A for enabling a return to a dashboard of the digital companion application, an interview icon 210B for enabling navigation to an interview page of the digital companion application, a call schedule icon 210C for enabling navigation to a call schedule page of the digital companion application, a previous call icon 210D for enabling navigation to a previous call page of the digital companion application, a notification setting icon 210E for enabling navigation to a notification setting page of the digital companion application, a skill icon 210F for enabling navigation to a skill page of the digital companion application, a user (elder) profile icon 210G for enabling navigation to an elder profile page of the digital companion application, and a contact icon 210H for enabling navigation to a contact page of the digital companion application. The call schedule icon 210C is highlighted to indicate that the user interface 204A shown in FIG. 2A corresponds to the call schedule page 212A.

The call schedule page 212A provides an interactive calendar 214 that enables users to configure both call frequency and skill-based engagement parameters. Call frequency can be granularly defined at an hourly level for each day of the week, allowing precise scheduling aligned with user preferences and routines. Skill assignments are managed through modular controllers 216A, 216B, 216C, which can be dynamically inserted into any time slot within the calendar. The controllers 216A, 216B, 216C allow adaptive skill mapping based on user availability, leveraging synchronization with external calendar data sources for conflict resolution and real-time updates.

In some implementations, the call schedule page 212A includes a header section with toggle elements 218A, 218B that activate direct communication channels between the AI-based companion and the user device 202, or vice versa. When enabled, the direct-call functionality computationally triggers the companion engine (e.g., companion engine 116, described with reference to FIGS. 1A-1C) to initiate proactive interactions using the preselected skills for scheduled calls. The skill-based mechanism (e.g., using the skill invocation engine 148, described with reference to FIG. 1C) employs low-latency signaling and context-aware invocation protocols to ensure seamless proactive and lively engagement, reducing repetition of topics and enhancing the perceived responsiveness of the AI model (e.g., AI model 118, described with reference to in FIGS. 1A and 1B).

FIG. 2B shows an example system 200B for managing skills of an artificial intelligence based digital companion, according to an implementation of the present disclosure. The example system 200B can include a user device 202 and a user interface 204B.

The user interface 204B can include a quick access bar 206 and a schedule interface 208. The quick access bar 206 can include a dashboard icon 210A for enabling a return to a dashboard of the digital companion application, an interview icon 210B for enabling navigation to an interview page of the digital companion application, a call schedule icon 210C for enabling navigation to a call schedule page of the digital companion application, a previous call icon 210D for enabling navigation to a previous call page of the digital companion application, a notification setting icon 210E for enabling navigation to a notification setting page of the digital companion application, a skill icon 210F for enabling navigation to a skill page of the digital companion application, a user (elder) profile icon 210G for enabling navigation to an elder profile page of the digital companion application, and a contact icon 210H for enabling navigation to a contact page of the digital companion application.

The skill icon 210F is highlighted to indicate that the user interface 204B shown in FIG. 2B corresponds to the skill page 212B that can be generated and managed by a skill registration engine (e.g., using the skill registration engine 146, described with reference to FIG. 1C). The skill page 212B can include a search bar 220 and a skill directory 222. As described herein, the skills page 212B specifies which skill providers (e.g., external data sources, websites and so forth) are available to the digital companion engine, e.g., to allow the digital companion engine to weave search-like insights in the otherwise AI operated conversation.

The skills directory 222 provides a structured interface displaying a comprehensive list of available skills, each represented with an icon, name, descriptive text, preview control, settings control, and a real-time status indicator. The skills encompass a wide range of functionalities, including breaking news delivery, sports updates, daily humor, smart device integration, family updates, story illustration, health monitoring, and conversational memory.

For example, the breaking news delivery skill provides streams of real-time headlines from trusted sources such as CNN, delivering updates on world events, politics, business, and entertainment. This skill uses external RSS or API feeds to inject verified content into conversations, ensuring factual accuracy and timeliness. As another example, sports update skill provides live scores, game schedules, and player statistics for selected teams. The skill leverages sports data APIs and event-driven triggers to deliver context-aware updates during calls, enhancing engagement for sports enthusiasts.

As another example, daily humor skill integrates humor content from Comedy Central, including jokes, one-liners, and short sketches. This skill uses curated content libraries and randomized selection algorithms to maintain freshness and avoid repetition. As another example, smart device integration skill connects to IoT-enabled refrigerators via manufacturer APIs (e.g., Samsung SmartThings), monitoring parameters such as door status, internal temperature, and food expiration dates. Event signals like door-open alerts can be surfaced in conversation as proactive reminders for meal preparation or energy conservation. These event signals can also be used to influence a conversation with the older adult and/or to send a notification or alert to a family member or other individual, e.g., if a sensor indicates that the older adult has not gotten out of bed after a specified amount of time.

As another example, family update skill synchronizes recent family news from social platforms such as Instagram to retrieve posts and updates from family members. The skill uses OAuth-based authentication and privacy-preserving filters to ensure secure and relevant content sharing. As another example, story illustration skill employs AI-powered visualization tools to convert user memories or anecdotes into visual representations. This skill uses generative models for image synthesis, enabling storytelling with personalized illustrations.

As another example, health monitoring skill integrates with medical IoT devices (e.g., blood pressure cuffs, glucose monitors) through secure APIs, providing real-time health metrics and alerts. Data streams are processed using anomaly detection algorithms to identify critical patterns and notify caregivers when necessary. As another example, family daily check-ins skill automates outreach to family members for daily status updates, aggregating responses into concise summaries for the user. The health monitoring skill uses asynchronous messaging protocols and natural language summarization to present updates conversationally. As another example, conversational memory skill maintains a persistent record of past interactions, enabling context continuity and personalization. The skill employs vector-based semantic indexing and retrieval mechanisms to recall relevant topics and user preferences during future sessions.

Each skill module is designed as a discrete service object that can be dynamically invoked during scheduled calls, enabling context-aware interactions. Notably, the system supports non-AI capabilities through skill blending, allowing external applications to participate in discussions by injecting relevant, real-time content. For example, current events are sourced from external feeds and pushed into the AI session rather than being generated by the AI model, ensuring factual accuracy and timeliness. Additionally, IoT integration is supported through device-linked skills, such as a smart refrigerator connection. In such implementations, sensor signals (e.g., door-open status, temperature thresholds) are captured via API endpoints and surfaced in conversation as proactive reminders—such as suggesting meal preparation or alerting the user to close the refrigerator door. This architecture leverages event-driven protocols and low-latency data pipelines to synchronize external device states with conversational flows, thereby enhancing engagement and utility for elderly users.

FIG. 2C shows an example data flow 200C for processing data for connecting user devices with companion engines for personalized communication sessions, according to an implementation of the present disclosure. In some implementations, the example data flow 200C occurs within the server system 102 of FIGS. 1A and 1B. The server system is configured to receive user data 232, which can be provided during a personalized communication with the user and included in a user communication request, as previously described with reference to FIG. 1A. The server system is configured to extract one or more features 234 from the user data 232. For example, the server system can extract extracted feature data 236 such as a profile 236A, condition 236B, sentiment 236C, user history 236D, or any other such data that are relevant to optimize the personalized communication with the user interacting with the user system.

The server system can process extracted feature data 236 using one or more processors and machine learning algorithms to identify relevant information for initiating and maintaining personalized communication with a user through an interactive interface. Leveraging the companion engine, the system performs topic extraction 238 by applying natural language processing (NLP) and semantic clustering techniques to the extracted feature data 236. The resulting topics can be categorized into critical topics 240A, which require immediate attention, and optional topics 240B, which provide supplementary engagement. The extracted topics 240C are transmitted to a prompt generation module 242, which constructs prompts using rule-based logic and contextual embeddings derived from user-specific data. The prompt generation process incorporates a context 244 that includes historical interactions, recent updates, and agenda priorities to ensure relevance. The prompt can be transmitted to an AI model 246, which is trained to produce conversational outputs aligned with user goals, which are handled by the companion engine to form the personalized communication with the user systems.

The AI model 246 is trained to generate personalized communication outputs for user engagement. The AI model 246 can be trained to include multiple specialized skills 248, which are implemented through modular sub-networks optimized for user-specific tasks. The skills 248 can include ability to conduct communication about news received from news sources identified as being of interest for the user according to the user profile. The skills 248 can include ability to conduct communication about sport updates and provide information related to sports identified as being of interest for the user according to the user profile. The skills 248 can include ability to conduct communication tailored to improve a mood of the user based on an identified sentiment for example by presenting jokes. The skills 248 can include ability to conduct communication related to lifestyle and diet by receiving updates from appliances, such as smart refrigerators or smart cooking appliances. The skills 248 can include ability to conduct communication related to life events and family updates determined from processing captured pictures, videos, and data extracted from social platforms. The skills 248 can include ability to conduct communication related to received medical user data associated with ongoing conditions 236B, sentiment 236C, and medical treatment plans derived from the user profile 236A. The skills 248 can include ability to adjust communication based on the conversation memory by reviewing past conversations and interviews to include meaningful topics for the user in the personalized communication.

The AI model 246 can be trained using training data that can be tailored for each of the skills. The training data includes structured medical records, conversational datasets, and compliance guidelines to ensure accuracy and regulatory adherence. by leveraging advanced natural language processing (NLP) and deep learning techniques. The AI model 246 can apply NLP as a set of computational methods to understand, interpret, and generate human language, incorporating processes such as tokenization, syntactic parsing, semantic analysis, and context modeling. The AI model 246 can apply NLP to comprehend user inputs, extract relevant user-related information, and formulate real time responses that are linguistically coherent and contextually appropriate. The AI model 246 can apply deep learning techniques, implemented through architectures such as transformer-based models (e.g., BERT, GPT, T5), recurrent neural networks (RNNs) like long short-term memory and gated recurrent unit, and sequence-to-sequence frameworks, which facilitate learning of complex patterns from large-scale user datasets and conversational corpora. The included deep learning techniques support multi-task learning, where specialized sub-networks handle symptom assessment, medication reminders, and motivational dialogue. The AI model 246 can apply deep learning techniques with attention mechanisms to prioritize critical user condition indicators and contextual cues, ensuring that generated outputs are both clinically relevant and personalized.

The AI model 246 uses contexts 244 (contextual embeddings) derived from user profiles, recent user updates, and agenda topics to produce responses that are both clinically relevant and empathetic. The AI model 246 includes a symptom assessment functionality that enables dynamic questioning and interpretation of user-reported conditions, while medication reminder modules integrate scheduling logic and dosage verification. Motivational dialogue components employ reinforcement learning strategies, including reinforcement learning with human feedback (RLHF), to maintain user engagement and adherence to different (social, emotional, and treatment) plans. The AI model 246 operates within a secure inference environment, utilizing encrypted communication channels and authenticated APIs to transmit outputs to the user device, ensuring confidentiality and integrity of sensitive health information.

Referring to FIG. 2D, the digital companion engine described herein has an open framework that allows it to engage seniors with more advanced technologies when that cohort becomes more tech-savvy. For example, the digital companion engine may initially engage with a senior over the telephone, e.g., by the digital companion engine initiating a telephone call. However, the companion engine is also configurable to specify which modalities an elder is able to or wants to use. For example, the companion engine can be configured to text with an elder, to communicate with the elder via an application (“app”) and app notifications, and so forth. The digital companion engine can also be “paired” with various types of devices, e.g., smart watches, remote patient monitoring (RPM) biometric device, motion sensors, smart devices, dedicated pods, cameras, robots, and so forth, to enable the digital companion engine to receive transmissions (e.g., data, communications and so forth) from those devices and to transmit communications (e.g., for the senior) to those devices, e.g., when the device is configured to receive communications. Generally, pairing devices includes establishing a secure, trusted wireless link (usually Bluetooth) by exchanging information, so they recognize and can communicate with each other easily in the future, allowing data sharing. Once paired, they store each other's info, bypassing the setup process for future connections. In some implementations, a first device can be identified as a primary communication device and a second device can be identified as a backup device. In response to determining that the companion engine cannot establish communication through the primary device, for example, if a battery level is low or a connection status indicates limited connectivity level, the companion engine can automatically switch to the backup device to initiate a scheduled companion communication session.

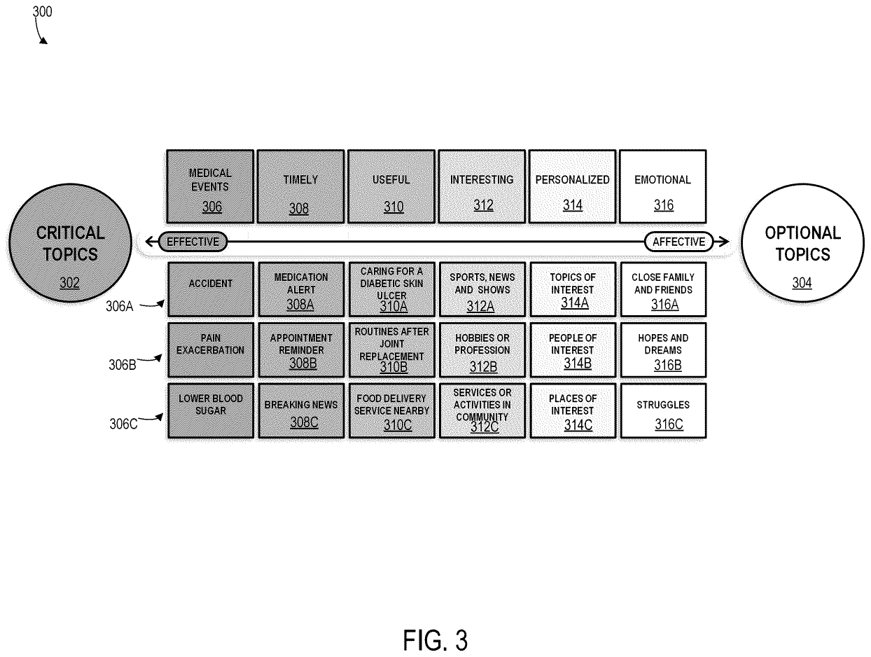

FIG. 3 is a block diagram illustrating an example data ranking 300, according to an implementation of the present disclosure. The example data ranking 300 ranges between critical topics 302 and optional topics 304 that can be included in a personalized communication with a user operating a user system. The critical topics 302 can be included to make the personalized communication more effective. The optional topics 304 can be included to make the personalized communication more affective.

Critical topics 302 represent high-priority subjects essential for effective user engagement and include medical events 306, such as accidents 306A, pain exacerbation 306B, and lower blood sugar 306C. Additional critical topics 302 include timely topics 308, such as medication alerts 308A, appointment reminders 308B, and breaking news 308C, as well as useful topics 310, such as caring for a diabetic skin ulcer 310A, routines after joint replacement 310B, and food delivery service nearby 310C.

Optional topics 304, which enhance affective engagement, include interesting topics 312, such as sports, news and shows 312A, hobbies or profession 312B, and services or activities in the community 312C. Other optional topics 304 can include personalized topics 314, such as topics of interest 314A, people of interest 314B, and places of interest 314C. Other optional topics 304 can include emotional topics 316, such as close family and friends 316A, hopes and dreams 316B, and struggles 316C. The structured data ranking 300 ranges between critical topics 302 and optional topics 304 enables the system to prioritize critical user-related subjects while incorporating optional, emotionally engaging topics to maintain user motivation and adherence to communication agendas.

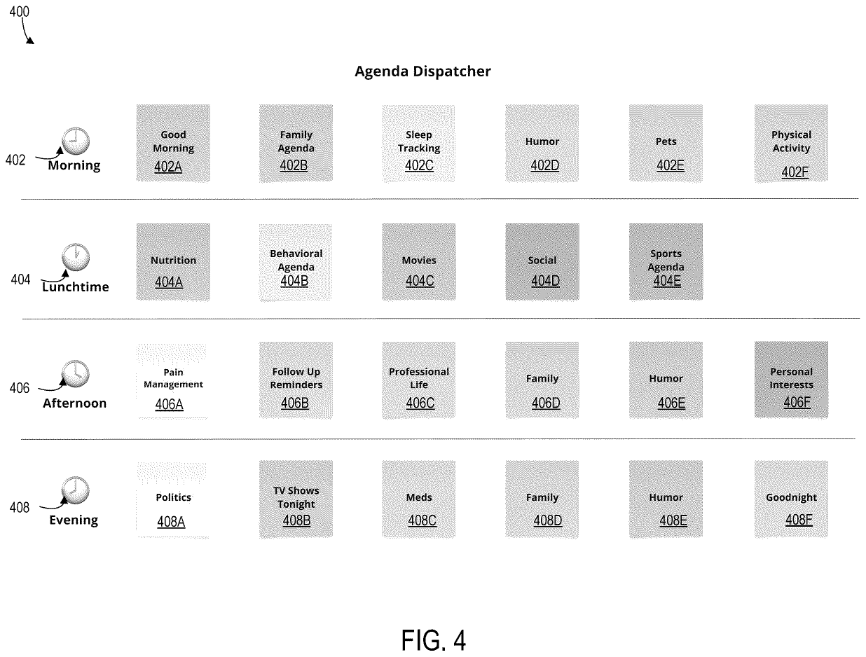

FIG. 4 is a block diagram illustrating an example agenda dispatcher 400, according to an implementation of the present disclosure. The agenda dispatcher 400 can be used to organize user interaction topics across different times of the day to enable structured and context-aware communication. The agenda dispatcher 400 can be divided into four-time segments: morning 402, lunchtime 404, afternoon 406, and evening 408, each containing multiple topic blocks.

In the morning, topics include good morning 402A, family agenda 402B, sleep tracking 402C, humor 402D, pets 402E, and physical activity 402F. Lunchtime topics include nutrition 404A, behavioral agenda 404B, movies 404C, social 404d, and sports agenda 404E. Afternoon topics focus on user and engagement, including pain management 406A, follow-up reminders 406B, professional life 406C, family 406D, humor 406E, and personal interests 406F. Evening topics include politics 408A, shows tonight 408B, medicaments 408C, family 408D, humor 408E, and goodnight 408F. The structured scheduling approach ensures that interactions are timely, relevant, and personalized, balancing critical user-related topics with affective and motivational content throughout the day. For each of these topics, the AI-based digital companion engine generates content (e.g., for an interaction with the elder) by identifying whether the topic is AI-based, is skilled-based or is both AI and skill based. For those topics that are AI-based, the AI-based digital companion engine generates and transmits a prompt the AI model. For those topics that are skilled-based, the AI-based digital companion engine requests from a skill provider system information related to the topic. And for topics that are based on both, the AI-based digital companion engine retrieves information from the skill provider system and uses that retrieved information in generating a prompt for the AI model.

The example agenda dispatcher 400 can be dynamically reorganized to personalize user interactions based on individual plans, schedules, and significant life events (identified by the user or people associated to the user, such as family and friends). For instance, morning topics such as good morning 402A and physical activity 402F can be replaced or supplemented with medication reminders aligned with prescribed dosing times or early-day therapy sessions. Lunchtime topics like nutrition 404A and behavioral agenda 404B can be adapted to include dietary guidance for managing chronic conditions or prompts for mid-day medicament intake. Afternoon topics such as pain management 406A and follow-up reminders 406B can be prioritized for users undergoing rehabilitation or monitoring post-surgical recovery. Evening topics like medication 408C and goodnight 408F can incorporate reminders for nighttime medications or relaxation techniques to improve sleep quality. Additionally, the agenda can integrate personalized prompts for significant life events—such as birthdays, anniversaries, or family milestones—within affective categories like family 402B/408D or personal interests 406F, ensuring emotional engagement alongside clinical adherence. The adaptive scheduling approach leverages user-specific data to deliver timely, relevant, and context-aware interactions.

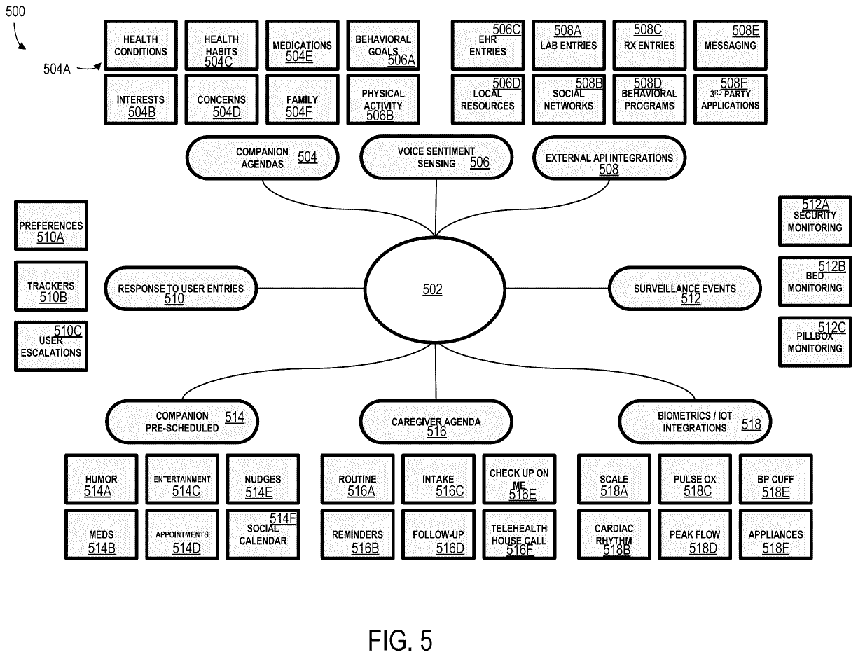

FIG. 5 is a block diagram illustrating an example data retrieval system 500, according to an implementation of the present disclosure. The example data retrieval system 500 includes a server system 502 that orchestrates data collection, processing, and communication for personalized user communication. The server system 502 can be coupled to multiple data sources that provide foundational inputs, including companion agendas 504, voice sentiment sensing 506, external API integrations 508, response to user entries 510, surveillance events 512, companion scheduled events 514, service provider agenda 516, and biometrics or IoT integrations 518.

The companion agendas 504 can be defined based on user conditions 504A, interests 504B, user habits 504C, concerns 504D, medications 504E, and family 504F. The user conditions 504A are retrieved and considered for identifying critical topics such as symptom monitoring and emergency alerts for personalized user communication. The user habits 504C are retrieved and considered to guide lifestyle recommendations. Discussions about the prescribed medications 504E can enable timely reminders and adherence tracking. The inclusion of family topics 504F adds affective engagement.

The voice sentiment sensing 506 can be defined relative to behavioral goals 506A, physical activity 506B, record entries 506C, and local resources 506D. The behavioral goals 506A, support motivational dialogue. Discussions about the physical activity 506B influences prompts for exercise and mobility. The server system 502 integrates voice sentiment sensing 508 to detect emotional states and adjust tone, and external API integrations for interoperability with data processing systems and IoT devices.

The external API integrations 508 can include lab entries 508A, social networks 508B, prescription-related data records 508C, behavioral programs 508D, messaging 508E, and third-party applications 508F. The prescription-related data records are critical for personalized communication facilitating the server system 502 to generate timely event (e.g., meetings, appointments, show times or medication) reminders, provide adherence nudges, and adjust conversational topics based on scheduled skills.

The response to user entries 510 can be fine-tuned based on preferences 510A, trackers 510B, and users escalations 510C. The surveillance events 512 can be identified based on sensor data received from user monitoring systems, such as security monitoring 512A, bed monitoring 512B (to monitor sleep patterns), pillbox monitoring 512C (to assist with medication protocol) and other house smart assets (e.g., refrigerator, microwave, oven, or other to monitor and assist with behavior patterns). The companion scheduled events 514 can include humor resources 514A, medications 514B, entertainment 514C, appointments 514D, nudges 514E, and social calendar 514F.

The service provider agenda 516 can be defined based on routines 516A, reminders 516B, intake 516C, follow-ups 516D, checkups 516E, and house call 516F. The biometrics or IoT integrations 518 can include data received from scaling systems 518A, cardiac rhythm monitors 518B, pulse oximeters 518C, peak flow monitors 518D, blood pressure cuffs 518E, and other appliances 518F. The biometrics or IoT integrations 518 supply continuous user metrics, which influence real-time prompts for well-being assessment and schedule and/or goal adherence. Together, the components coupled to the server system 502 enable secure, context-aware, and adaptive communication tailored to the user's medical needs and emotional well-being.

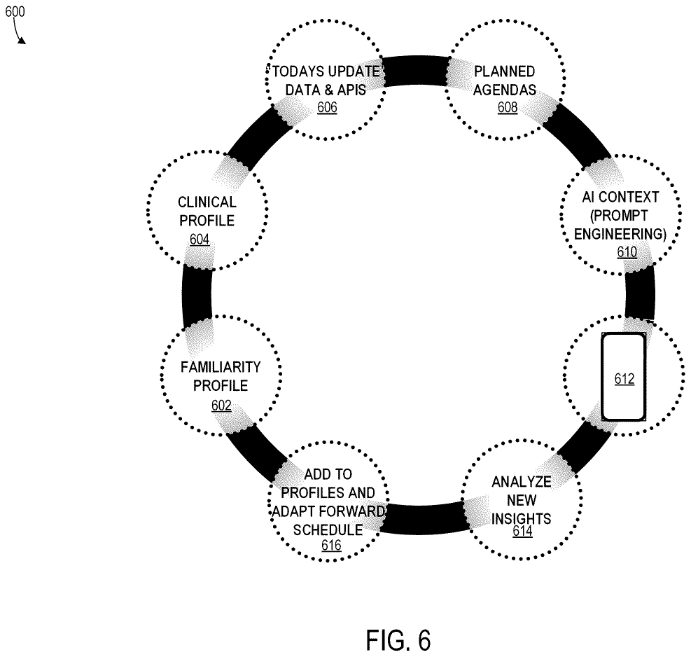

FIG. 6 shows an example data flow 600 for generating personalized interactions, according to an implementation of the present disclosure. Before contact is initiated, the server system prepares the AI interaction by combining four initial elements: the familiarity profile 602, the clinical profile 604, recently updated data and APIs 606, and the planned agendas 608.

The familiarity profile 602 captures historical engagement patterns and user preferences. The clinical profile 604 includes medical conditions, treatment plans, and medication schedules. The updated data and APIS 606 provide near-time user metrics and external resource updates. The planned agendas 608 define upcoming topics for discussion. The initial elements are merged within the AI context (by a prompt generation module) 610, where contextual embeddings and compliance rules are applied to generate a structured prompt for the conversational engine. The prompt can be transmitted to the user system (e.g., user communication device) 612 for real-time interaction.

In response to completing each conversation, the system performs an analysis of new insights 614, extracting previously unknown information from the interaction transcript using NLP and machine learning techniques. The insights are converted into AI-prompt form and dispatched to augment the familiarity profile, update the clinical profile, and influence the agenda schedule, which is managed by the add to profiles and adapt forward schedule 616 module. The cyclical process ensures that each interaction between a user (elder) and a server system improves personalization, clinical relevance, and engagement quality during personalized communications.

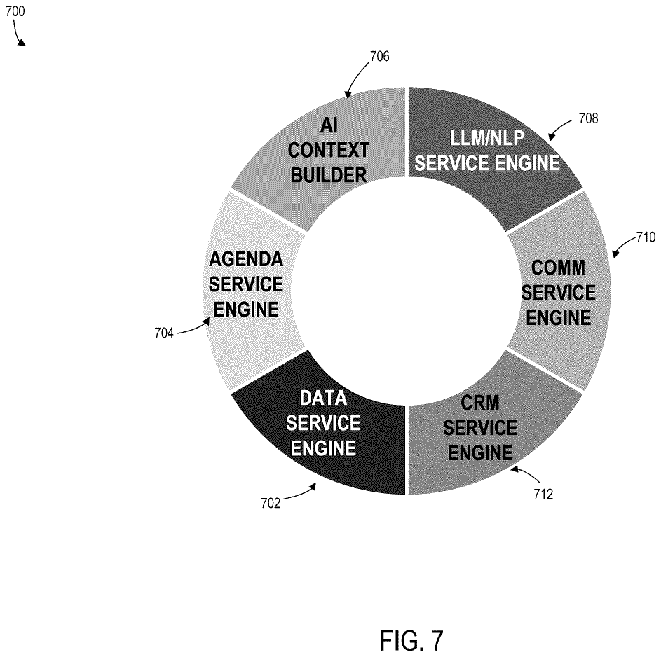

FIG. 7 is a block diagram illustrating an example system core architecture 700 for executing a companion engine for personalized communication sessions, according to an implementation of the present disclosure. The example system core architecture 700 includes interconnected service engines, each responsible for a distinct functional layer within a server system (e.g., server system 102 described with reference to FIGS. 1A and 1B). In particular, the example system core architecture 700 includes a data service engine 702, an agenda service engine 704, an AI context builder 706, an LLM/NLP service engine 708, a communication service engine 710, and a CRM service engine 712.

The data service engine 702 manages the sourcing and delivery of data used to influence agenda services and interaction payloads in near real-time. The data service engine 702 functions as the backbone of the system by sourcing, processing, and delivering relevant data in near real-time to support personalized interactions. The data service engine 702 aggregates information from multiple sources (as described with reference to FIG. 5), including user profiles, historical interaction logs, external APIs, and contextual signals such as location and time. The raw data is normalized, structured, and enriched with metadata to ensure is actionable for downstream services. Once processed, the data service engine 702 streams or pushes the data to the agenda service engine 704 and AI context builder 706, enabling these components to generate dynamic conversational agendas and interaction payloads that reflect most current information. By providing timely and contextually relevant data, the data service engine 702 directly influences the tone, content, and personalization of interactions, ensuring that each engagement is adaptive and highly tailored to the individual.

The agenda service engine 704 establishes the schedule, content, tone, and personal adaptation of daily conversational agendas. For example, the agenda service engine 704 is responsible for orchestrating the structure and personalization of daily conversational agendas by defining the schedule, content, tone, and adaptive elements for each interaction. The agenda service engine 704 begins by analyzing user-specific data, such as preferences, historical behavior, and contextual signals, to determine the optimal timing and sequencing of agenda items. The agenda service engine 704 curates the content to align with the user's interests and priorities, ensuring relevance and engagement. Additionally, the agenda service engine 704 adjusts the tone of communication formal, casual, or empathetic based on the user's profile, current events, and interaction history. The tone of communication can be formal, casual, or empathetic. To achieve personal adaptation, the agenda service engine 704 dynamically modifies agendas in response to real-time inputs, such as changes in user availability or external events, creating a fluid and highly personalized conversational experience.

The AI context builder 706 prepares the interaction payload and performs prompt engineering for the AI service prior to each engagement. The AI context builder 706 is configured to prepare the interaction payload and perform prompt engineering for the AI service before each communication engagement, ensuring that conversations are contextually accurate and personalized. The AI context builder 706 begins by aggregating relevant inputs from the data service engine 702 and the agenda service engine 704, including user-specific data, agenda details, and real-time contextual signals. Using the aggregated information, the AI context builder 706 constructs a structured payload that defines the objectives, tone, and constraints of the upcoming interaction. Additionally, the AI context builder 706 applies prompt engineering techniques to optimize how instructions and contextual cues are presented to the underlying AI model, improving response quality and alignment with user expectations. By dynamically tailoring prompts and payloads for each interaction, the AI context builder 706 ensures that the AI service operates with precise context, delivering coherent, adaptive, and highly personalized conversational experiences.

The LLM/NLP service engine 708 governs the core artificial intelligence and natural language processing functions that execute the interaction logic within the system. The LLM/NLP service engine 708 serves as the operational layer where large language models (LLMs) and speech services are deployed to interpret prompts, generate responses, and manage conversational flow. The LLM/NLP service engine 708 receives the structured payload and optimized prompts prepared by the AI Context Builder 706, then applies advanced language understanding and generation capabilities to produce coherent, contextually relevant outputs. Additionally, the LLM/NLP service engine 708 integrates speech synthesis and recognition components when voice-based interactions are required, ensuring seamless multimodal communication. By orchestrating the AI and NLP processes, the LLM/NLP service engine 708 ensures that each interaction is accurate, adaptive, and aligned with the user's personalized agenda, forming the intelligence backbone of the system's conversational experience.

The communication service engine 710 is responsible for managing the transport and delivery of interactions across all supported communication channels, ensuring seamless connectivity between the system and the end user. The communication service engine 710 acts as the integration layer that translates interaction outputs from the LLM/NLP Service Engine 708 into channel-specific formats, such as voice for phone calls or text for messaging platforms. The communication service engine 710 handles protocol compatibility, message routing, and session management to maintain reliability and consistency across diverse communication mediums. Additionally, the communication service engine 710 supports real-time transmission and synchronization, enabling interactions to occur without latency while preserving context across channels. By providing the transport functionality, the communication service engine 710 ensures that personalized, AI-driven engagements are delivered effectively through the user's preferred communication method. In some implementations, the communication service engine 710 triggers activation of user monitoring systems or activation of second user devices to initiate a support operation. For example, a light or sound alert coupled to a pill box can be activated to provide a reminder for a scheduled medication administration. As another example, a healthcare monitoring system (e.g., blood pressure cuff) can be activated to repeat a measurement in response to detecting an abnormal sensor data outside of an expected range for the user.

The CRM service engine 712 is responsible for maintaining and expanding the system's familiarity with each individual over time, ensuring continuity and personalization in future interactions. The CRM service engine 712 supports continuity and personalization in communications by storing and updating user-specific data, such as preferences, historical interactions, behavioral patterns, and contextual insights gathered during engagements. The CRM service engine 712 continuously refines the user profile through machine learning and adaptive algorithms, allowing the system to anticipate needs and deliver increasingly relevant and personalized experiences. By leveraging accumulated knowledge, the CRM service engine 712 enables the system to maintain a consistent tone, recognize prior conversations, and adapt agendas based on evolving user interests or circumstances. The long-term memory function is critical for creating a seamless, relationship-driven interaction model that feels natural and tailored to the individual. The components of the example system core architecture 700 form a cohesive architecture enabling dynamic, personalized, and multi-channel user engagement for optimizing personalized communications.

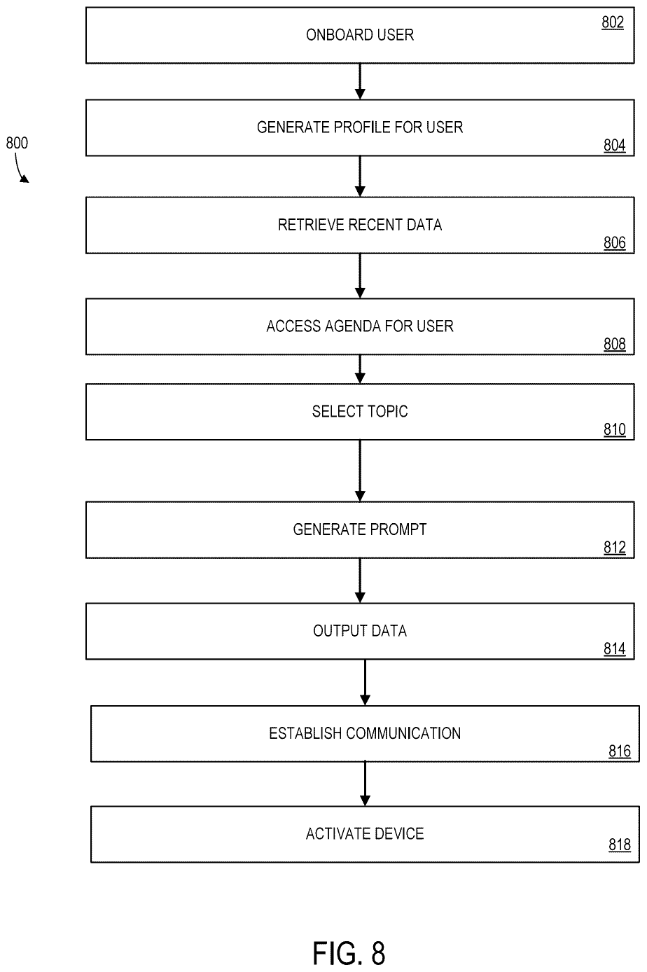

FIG. 8 is a flowchart illustrating an example of a computer-implemented method 800 for executing a companion engine for personalized communication sessions, according to an implementation of the present disclosure. For clarity of presentation, the description that follows generally describes computer-implemented method 800 in the context of the other figures in this description. However, it will be understood that computer-implemented method 800 can be performed, for example, by any system, environment, software, and hardware, or a combination of systems, environments, software, and hardware, as appropriate. In some implementations, various steps of computer-implemented method 800 can be run in parallel, in combination, in loops, or in any order.

At 802, a user is onboarded for generating personalized communications using an AI-based companion. Onboarding the user can include receiving, from a user communication device (e.g., user device 104A, described with reference to FIG. 1B) one or more Onboarding can include capturing electronic initials to accept Terms of Use of the personalized companion, ensuring compliance and consent with security settings. Onboarding can include an introduction call to caregivers, followed by scheduling and dispatching interviews. The interviews are dynamically assigned to different people including the user and connected to the user (e.g., family members, friends, and caregivers), allowing distributed data collection for personalization of user datasets. Additionally, the onboarding workflow provides an option for one or more connected people to record personalized audio messages for the senior user, which can be played at the start of the introductory call to establish familiarity and trust. Once interviews are completed, the user configures a call schedule (as referenced in FIG. 2A), defining frequency and skill preferences for future sessions. The onboarding process may also include an introduction call to the AI-based companion for the senior user, creating an initial engagement experience. Each of the onboarding steps is treated as an event type within the system architecture, enabling event-driven orchestration, audit logging, and real-time status tracking. Collectively, the onboarding pipeline ensures secure authentication, personalized data acquisition, and a smooth transition into AI-assisted communication tailored to the cognitive and emotional needs of elderly users. From 802, computer-implemented method 800 proceeds to 804.

At 804, a profile for the user is generated based on the one or more received answers. A profile for the user is generated by analyzing one or more received answers and compiling them into a structured representation of the individual's preferences, interests, and behavioral patterns. The user profile includes categories such as hobbies and interests (e.g., sports, art, profession, or gossip following), as well as lifestyle indicators (as described with reference to FIG. 3) that help personalize future interactions. In addition to capturing the user preferences, generating a user's profile can include pairing or establishing connections to external services and platforms, such as linking a social media profile or integrating a cable or streaming television lineup, to enrich the personalization layer. The user profile includes information that can be applied to generate an affective conversation that is contextually relevant, adaptive, and aligned with both the user's personal interests and the current user status, creating a highly tailored and engaging experience. The user profile can also include known clinical information, such as user conditions and ongoing plans and schedules including fitness schedules, social engagement schedules and timing of prescribed medication and other useful information defining critical topics. In some implementations, generating the user profile includes generating multiple agendas distributed according to a time schedule, as described with reference to FIG. 4). From 804, computer-implemented method 800 proceeds to 806.

At 806, recent data is retrieved from one or more sources (as described with reference to FIGS. 1A, 1B, and 5), by one or more application program interfaces (APIs). The recent data associated with the user can include data having occurred within a threshold amount of time (e.g., less than a year or a month). The recent data can include medical events timely information and/or recently mentioned data received from monitoring systems including monitoring devices configured to support user conditions including user safety. The recent data associated with the user can be retrieved using a digital identifier by accessing a computing system associated with the digital identifier, the computing system hosting the one or more data records through an application interface that is different from another application interface associated with one or more other data records associated with a different computing system associated with another digital identifier. From 806, computer-implemented method 800 proceeds to 808.

At 808, an agenda for the user is accessed from a centralized database that stores structured schedules and conversation plans tailored to individual needs. The agenda includes both critical topics, such as health-related updates or medication reminders, and optional topics designed to enrich engagement, such as hobbies, entertainment, or social interests. The agenda can include service provider-added agenda items, supporting insertion of personalized discussion points or reminders based on real-time observations or evolving user needs. From 808, computer-implemented method 800 proceeds to 810.

At 810, a topic for interaction with the user is selected by synthesizing information from three key sources: the generated user profile, recent data updates, and the predefined agenda. Selecting the topic can include evaluation of the user's profile, which contains personal interests, lifestyle preferences, and historical engagement patterns, to identify subjects that are likely to resonate and maintain engagement. Selecting the topic can include adjustments based on near-time data, such as current events, user updates, or contextual signals, to ensure relevance and timeliness. Selecting the topic can include adjustments relative to critical user-related topics and optional conversational items, as well as service provider-added points. By prioritizing topics that align with both the user's preferences and the agenda requirements, the system ensures that each interaction is purposeful, personalized, and contextually appropriate, fostering a meaningful and adaptive conversational experience. Additional topics can be extracted by receiving, through the communication channel, input data from the user device of the user, generating of transcript of the communication based on the received input data, and identifying, based on the generated transcript, one or more attributes of the user. From 810, computer-implemented method 800 proceeds to 812.

At 812, a prompt for the interaction is generated by combining the selected topic with its contextual details derived from the user's profile and recent data updates (as described with reference to FIG. 2C) to maintain conversations focused on recent and relevant events. The prompt generation can include an identification of the core subject matter from the agenda and enrichment with personalized context, such as the user's interests, communication style, and historical engagement patterns stored in the profile. The prompt generation can include identification of context related to near-time information, including clinical updates, environmental cues, or relevant external events, to ensure the prompt is timely and meaningful. Using these inputs, the system applies prompt engineering techniques to structure the instructions for the AI model, defining tone, objectives, and constraints for the conversation. The prompt generation can include live content incorporation for continuous adjustment to user current physical and mental state for optimized support through personalized conversations. From 812, computer-implemented method 800 proceeds to 814.

At 814, the prompt is processed, by the AI model, to generate conversational output relevant for a goal of the user. The conversational output can be formatted to support proactive conversations including text messaging, regular calls (audio communications), and/or audio-video communications. The AI model can be trained to provide adjustable communication outputs according to a set of skills (as described with reference to FIG. 2B). The prompt is processed by the AI model through a series of natural language understanding and generation steps to produce conversational output that aligns with the user's goals. The AI model interprets the structure of the prompt and embedded instructions using advanced language processing algorithms. Prompt processing includes a semantic analysis to understand the user's intent, such as reinforcing medication adherence, promoting cognitive engagement, or addressing emotional well-being. The AI model generates responses that are contextually relevant, empathetic, and clinically appropriate, ensuring that the conversation remains aligned with both the user's personal interests and their health objectives. By leveraging the engineered prompt and its contextual cues, the AI delivers output that is not only coherent and engaging but also strategically designed to support the user's care plan and overall well-being. From 814, computer-implemented method 800 proceeds to 816.

At 816, an additional communication is established with an additional device. The external device can include a service provider's smartphone, a monitoring device, or a medical device. The additional communication can be established through secure communication protocols designed to prevent malicious interference and ensure data integrity. When a trigger event occurs, such as an abnormal vital sign detected by a wearable monitor or a service provider-added agenda item requiring immediate attention, the system activates encrypted transport channels using standards like TLS or VPN tunneling. Authentication mechanisms, including multi-factor verification and token-based access control, can be applied to validate both the system and the external additional device before any data exchange begins. Furthermore, session keys can be dynamically generated for each interaction to prevent replay attacks, and continuous monitoring is implemented to detect anomalies or unauthorized access attempts. The security measures ensure that communications remain confidential, tamper-proof, and compliant with privacy data protection regulations, enabling safe and reliable coordination between the user, caregivers, and medical systems. From 816, computer-implemented method 800 proceeds to 818.

At 818, the additional device is activated through activation signal transmission over the secure communication protocols. For example, if a wearable monitoring device reports an abnormal heart rate, the user system can automatically initiate a communication with the server system and generate a notification or voice call to a service provider (e.g., the caregiver's device) to facilitate a timely connection between the user device and a service provider system. As another example, if the user's agenda includes a scheduled audio/video session, the user system can trigger a video call setup connecting a service provider system (e.g., the caregiver's device or connect to a medical device) to facilitate access to the service offered by the service provider within a graphical user interface of the user device. Triggering the video call setup through the caregiver's device can include transmission of relevant user data that can be ranked relative to how critical an associated recent event was classified as being to facilitate streamed interventions for timely treating critical conditions. As another example, if the user's agenda includes a scheduled medicament administration a medical device configured for administering the treatment can be activated (e.g., by modifying a state of an on/off switch) and an alert (e.g., light alert, acoustic alert or haptic alert) can be started. The activations ensure timely intervention and seamless coordination between the user and service provider systems, enhancing safe and secure communications. After 818, computer-implemented method 800 can return to any of the previous operations. For example, attributes of the user identified during a conversation can be used to update the profile of the user during the conversation or after completing the conversation. As another example, method 800 can return to the topic selection for generating another prompt for the AI engine, with a second topic specifying a request for another interaction with the user and further specifying the updated profile.

FIG. 9 is a block diagram of an example computing system 900 used to provide computational functionalities associated with described algorithms, methods, functions, processes, flows, and procedures described in the present disclosure, according to some implementations of the present disclosure. The illustrated computer 902 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 902 can include input devices such as keypads, keyboards, and touch screens that can accept user information. Also, the computer 902 can include output devices that can convey information associated with the operation of the computer 902. The information can include digital data, visual data, audio information, or a combination of information. The information can be presented in a graphical user interface (UI) (or GUI).

The computer 902 can serve in a role as a user, a network component, a server, a database, a persistency, or components of a computer system for performing the subject matter described in the present disclosure. The illustrated computer 902 is communicably coupled with a network 924. In some implementations, one or more components of the computer 902 can be configured to operate within different environments, including cloud-computing-based environments, local environments, global environments, and combinations of environments.

At a high level, the computer 902 is an electronic computing device operable to receive, transmit, process, store, and manage data and information associated with the described subject matter. According to some implementations, the computer 902 can also include, or be communicably coupled with, an application server, an email server, a web server, a caching server, a streaming data server, or a combination of servers.

The computer 902 can receive requests over network 924 from a user application (for example, executing on another computer 902). The computer 902 can respond to the received requests by processing the received requests using software applications. Requests can also be sent to the computer 902 from internal users (for example, from a command console), external (or third) parties, automated applications, entities, individuals, systems, and computers.

Each of the components of the computer 902 can communicate using a system bus 904. In some implementations, any or all the components of the computer 902, including hardware or software components, can interface with each other or the interface 906 (or a combination of both), over the system bus 904. Interfaces can use an application programming interface (API) 914, a service layer 916, or a combination of the API 914 and service layer 916. The API 914 can include specifications for routines, data structures, and object classes. The API 914 can be either computer-language independent or dependent. The API 914 can refer to a complete interface, a single function, or a set of APIs.

The service layer 916 can provide software services to the computer 902 and other components (whether illustrated or not) that are communicably coupled to the computer 902. The functionality of the computer 902 can be accessible for all service users using this service layer. Software services, such as those provided by the service layer 916, can provide reusable, defined functionalities through a defined interface. For example, the interface can be software written in JAVA, C++, or a language providing data in extensible markup language (XML) format. While illustrated as an integrated component of the computer 902, in alternative implementations, the API 914 or the service layer 916 can be stand-alone components in relation to other components of the computer 902 and other components communicably coupled to the computer 902. Moreover, any or all parts of the API 914 or the service layer 916 can be implemented as child or sub-modules of another software module, enterprise application, or hardware module without departing from the scope of the present disclosure.

The computer 902 includes an interface 906. Although illustrated as a single interface 906 in FIG. 9, two or more interfaces 906 can be used according to needs, desires, or particular implementations of the computer 902 and the described functionality. The interface 906 can be used by the computer 902 for communicating with other systems that are connected to the network 924 (whether illustrated or not) in a distributed environment. Generally, the interface 906 can include, or be implemented using, logic encoded in software or hardware (or a combination of software and hardware) operable to communicate with the network 924. More specifically, the interface 906 can include software supporting one or more communication protocols associated with communications. As such, the network 924 or the hardware of the interface can be operable to communicate physical signals within and outside of the illustrated computer 902.

The computer 902 includes a processor 908. Although illustrated as a single processor 908 in FIG. 9, two or more processors 908 can be used according to particular implementations of the computer 902 and the described functionality. Generally, the processor 908 can execute instructions and can manipulate data to perform the operations of the computer 902, including operations using algorithms, methods, functions, processes, flows, and procedures as described in the present disclosure.

The computer 902 also includes a database 920 that can hold data (for example, data 922) for the computer 902 and other components connected to the network 924 (whether illustrated or not). For example, database 920 can be an in-memory, conventional, or a database storing data consistent with the present disclosure. In some implementations, database 920 can be a combination of two or more different database types (for example, hybrid in-memory and conventional databases) according to particular implementations of the computer 902 and the described functionality. Although illustrated as a single database 920 in FIG. 9, two or more databases (of the same, different, or combination of types) can be used according to particular implementations of the computer 902 and the described functionality. While database 920 is illustrated as an internal component of the computer 902, in alternative implementations, database 920 can be external to the computer 902.

The computer 902 also includes a memory 910 that can hold data for the computer 902 or a combination of components connected to the network 924 (whether illustrated or not). Memory 910 can store any data consistent with the present disclosure. In some implementations, memory 910 can be a combination of two or more different types of memory (for example, a combination of semiconductor and magnetic storage) according to implementations of the computer 902 and the described functionality. Although illustrated as a single memory 910 in FIG. 9, two or more memories 910 (of the same, different, or combination of types) can be used according to implementations of the computer 902 and the described functionality. While memory 910 is illustrated as an internal component of the computer 902, in alternative implementations, memory 910 can be external to the computer 902.

The application 912 can be an algorithmic software engine providing functionality according to implementations of the computer 902 and the described functionality. For example, application 912 can serve as one or more components, modules, or applications. Further, although illustrated as a single application 912, the application 912 can be implemented as multiple applications 912 on the computer 902. In addition, although illustrated as internal to the computer 902, in alternative implementations, the application 912 can be external to the computer 902.

The computer 902 can also include a power supply 918. The power supply 918 can include a rechargeable or non-rechargeable battery that can be configured to be either user- or non-user-replaceable. In some implementations, the power supply 918 can include power-conversion and management circuits, including recharging, standby, and power management functionalities. In some implementations, the power-supply 918 can include a power plug to allow the computer 902 to be plugged into a wall socket or a power source to, for example, power the computer 902 or recharge a rechargeable battery.

There can be any number of computers 902 associated with, or external to, a computer system containing computer 902, with each computer 902 communicating over network 924. Further, the terms “user,” “user,” and other appropriate terminology can be used interchangeably, as appropriate, without departing from the scope of the present disclosure. Moreover, the present disclosure contemplates that many users can use one computer 902 and one user can use multiple computers 902.

The subject matter described in this specification can be implemented to realize one or more of the following advantages. The described approach advantageously provides a secure and interoperable architecture for simulating a personal companion for elderly users, enabling real-time interactions with companion engines. As one advantage, the system ensures confidentiality and integrity of sensitive user data by employing encrypted communication channels and authenticated APIs for all data exchanges. As another advantage, the architecture supports dynamic security adaptation, facilitating encryption strength and authentication protocols to adjust based on the sensitivity of the user's goal, thereby improving resilience against evolving threats. As another advantage, the described approach isolates contextual data from conversational outputs, reducing the attack surface and preventing leakage of protected user information. As a further advantage, secure session establishment through TLS-based protocols guarantees compliance with privacy regulations while maintaining uninterrupted connectivity. The described approach also enables cross-network interoperability without compromising security, providing a technical improvement over conventional systems that rely on static segmentation.

Existing technologies for delivering live, real-time user relevant content typically rely on centralized streaming platforms or provider-specific systems that enable users to access content through predefined channels. Some traditional systems often implement basic search functionality based on general categories or tags but lack advanced attribute-based matching for personalized experiences. Furthermore, current solutions are fragmented, with limited interoperability between provider networks, resulting in users needing to navigate multiple platforms to locate desired content. Personalization is minimal, as most systems do not dynamically adapt to individual user preferences and are not able access, e.g., in real-time, information about the user (e.g., information specifying that a refrigerator was not opened in the morning). Additionally, information about available live communications is often incomplete or outdated, leading to inefficiencies in connecting users with relevant service providers. The limitations of the existing technologies create a gap in delivering secure, seamless, and personalized real-time communication experiences leading to inefficient user support.

The described system overcomes the limitations of existing technologies by providing a secure and interoperable architecture that enables real-time user interactions for elderly users across segmented networks, e.g., without requiring an elderly user to prompt an AI system or otherwise initiate an interaction.

In contrast to conventional systems that lack personalization and suffer from fragmented data access, the described approach ensures confidentiality and integrity of sensitive user information through encrypted communication channels and authenticated APIs for all exchanges. The described approach further introduces dynamic security adaptation, allowing encryption strength and authentication protocols to adjust based on the sensitivity of the user's goal, thereby improving resilience against evolving security threats. By isolating contextual data from conversational outputs, the described system reduces the attack surface and prevents leakage of protected user information. Secure session establishment through TLS-based protocols guarantees compliance with privacy regulations while maintaining uninterrupted connectivity. Additionally, the described architecture enables cross-network interoperability without compromising security, delivering seamless, real-time adaptive communication supporting personalized care.

### Technical Improvements to Artificial Intelligence and Machine Learning Operation

In various embodiments, the disclosed system provides improvements to the operation of artificial intelligence and machine learning systems used for continuous, ambient user assistance.

Conventional AI-driven assistant systems typically rely on static models, predefined interaction triggers, or explicit user commands to initiate processing and generate outputs. Such systems often fail to adapt efficiently to individual users over time and generate redundant or low-relevance outputs that increase computational overhead and degrade system performance.

The disclosed system improves the operation of the AI itself by implementing machine learning models that maintain and continuously update user-specific behavioral representations derived from interaction data, contextual signals, and environmental sensing data. These representations function as adaptive model inputs that evolve over time, enabling the AI to modify its inference behavior without retraining global models or relying on fixed rule sets.

In certain embodiments, the system employs incremental or online learning techniques that update user-specific parameters in response to newly observed data generated during runtime operation of the system. The machine learning architecture is configured to distinguish between global model parameters, which capture population-level patterns learned during an initial training phase, and user-specific adaptive parameters, which are maintained separately for each individual user and updated continuously as new interaction, contextual, or sensing data is observed.

The global model parameters define a shared representational space, feature extraction logic, and inference structure that remain fixed during normal operation, thereby providing a stable baseline for model behavior. The user-specific adaptive parameters modify the operation of the global model by adjusting one or more of input feature weightings, embedding offsets, attention weights, thresholds, or decision boundaries based on observed user-specific behavior. This separation enables personalization to occur without modifying the underlying global model architecture.

During runtime, newly observed data is processed to generate incremental updates to the user-specific adaptive parameters using lightweight update operations, such as gradient updates constrained to a subset of parameters, statistical aggregation of recent observations, or reinforcement signals derived from user responses. Because these updates are limited in scope and do not require backpropagation through the full model, the system avoids repeated full-model retraining and associated computational cost.

By maintaining user-specific adaptive parameters independently of the global model, the system supports real-time learning that occurs continuously as interactions take place, rather than in batch retraining cycles. This enables the AI system to adapt inference behavior promptly in response to changes in user routines, preferences, or contextual patterns, while preserving stability and consistency across users.

The disclosed parameter separation further enables the system to scale personalization across a large number of users, as user-specific updates are isolated and do not propagate across the shared global model. As a result, updates for one user do not degrade or interfere with inference behavior for other users, and the system can perform personalization using limited computational and memory resources.

Accordingly, the use of incremental or online learning with separated global and user-specific parameters constitutes an improvement to the operation of the machine learning system itself by reducing retraining overhead, enabling continuous adaptation during runtime, improving inference responsiveness, and supporting scalable personalization without sacrificing model stability.

The disclosed AI further improves inference accuracy by using learned behavioral baselines as reference states during runtime. Rather than evaluating inputs against static thresholds, the machine learning models evaluate changes relative to individualized baselines, allowing the AI to distinguish meaningful deviations from normal variation. This approach improves signal-to-noise ratio in model outputs and reduces false positives generated by conventional anomaly detection systems.

In some embodiments, the AI integrates conversational context, temporal data, and historical interaction state into a unified feature representation used by the machine learning models during inference. This improves the internal statefulness of the AI system, enabling continuity across interactions and reducing incoherent or repetitive outputs that commonly arise in stateless or session-bound AI architectures.

Additionally, the disclosed system improves AI resource utilization by enabling passive inference scheduling, wherein the AI selectively performs inference operations based on learned relevance and temporal patterns, rather than continuously executing inference cycles or responding to every sensed event. This reduces unnecessary computation and network usage while preserving responsiveness to significant changes.

Accordingly, the disclosed techniques provide improvements to the functioning of artificial intelligence systems themselves, including improved adaptability without full retraining, more efficient personalization, improved anomaly discrimination, reduced computational overhead, and enhanced temporal coherence of AI outputs. These improvements arise from specific modifications to how machine learning models are structured, updated, and executed, rather than from the mere use of AI as a tool to perform conventional tasks.

Described implementations of the subject matter can include one or more features, alone or in combination.
Example 1. A computer-implemented method comprising: simulating, by a computer system, an artificial intelligence (AI)-based digital companion for a user, comprising:
onboarding the user by receiving, from a user device, one or more answers to one or more interview questions; based on the one or more received answers, generating a profile for the user; retrieving, from one or more external data sources by one or more application program interfaces (APIs), recent data associated with the user, with the recent data having occurred within a threshold amount of time; accessing, from a hardware storage device, an agenda for the user, the agenda comprising a plurality of topics to be addressed with the user; based on the generated profile, the recent data and the agenda, selecting a topic for an interaction with the user; generating a prompt, based on the topic for the interaction with the user and a context of the topic determined from the generated profile and the recent data; transmitting the prompt to an AI engine trained to generate a conversational output relevant to the user; receiving, through an API of the AI engine, output data specifying one or more instructions for interacting with the user; and establishing, through a communication channel with a user device of the user, a communication with the user in accordance with the one or more instructions.
Example 2. The computer-implemented method of the previous example, wherein the topic is a first topic and wherein the method further comprises: receiving, through the communication channel, input data from the user device of the user; generating of transcript of the communication based on the received input data; identifying, based on the generated transcript, one or more attributes of the user; responsive to the identifying, updating a profile of the user based on the one or more attributes; and transmitting a second topic to the AI engine, with the second topic specifying a request for another interaction with the user and further specifying the updated profile.
Example 3. The computer-implemented method of any of the previous examples, wherein the recent data associated with the user is retrieved using a digital identifier by accessing a computing system associated with the digital identifier, the computing system hosting the one or more data records through an application interface that is different from another application interface associated with one or more other data records associated with a different computing system associated with another digital identifier.
Example 4. The computer-implemented method of any of the previous examples, wherein retrieving the recent data associated with the user associated with the digital identifier comprises accessing authorization data associated with the digital identifier; and accessing, using the authorization data, a computing system hosting the one or more data records.
Example 5. The computer-implemented method of any of the previous examples, wherein retrieving the recent data associated with the user associated with the digital identifier comprises activating a sensor for initiating data collection in real time.
Example 6. The computer-implemented method of any of the previous examples, wherein the recent data associated with the user is constrained to a threshold time period defining topic currency.
Example 7. The computer-implemented method of any of the previous examples, comprising activating a device for generating an alert associated with the one or more instructions.
Example 8. The computer-implemented method of any of the previous examples, wherein the device comprises a medical assistance device and the alert associated with the one or more instructions corresponds to an event task.
Example 9. The computer-implemented method of any of the previous examples, comprising determining a context for the topic, wherein the context defines behavioral and user data associated to the topic.
Example 10. The computer-implemented method of any of the previous examples, comprising establishing, through an additional communication channel a communication between the user device of the user and a provider gateway system for rendering a visual representation of a service provider within a graphical user interface of the user device.
Example 11. A computer-implemented system, comprising: one or more computers; and one or more computer memory devices interoperably coupled with the one or more computers and having tangible, non-transitory, machine-readable media storing one or more instructions that, when executed by the one or more computers, perform one or more operations, comprising: simulating, by a computer system, an artificial intelligence (AI)-based digital companion for a user, comprising: onboarding the user by receiving, from a user device, one or more answers to one or more interview questions; based on the one or more received answers, generating a profile for the user; retrieving, from one or more external data sources by one or more application program interfaces (APIs), recent data associated with the user, with the recent data having occurred within a threshold amount of time; accessing, from a hardware storage device, an agenda for the user, the agenda comprising a plurality of topics to be addressed with the user; based on the generated profile, the recent data and the agenda, selecting a topic for an interaction with the user; generating a prompt, based on the topic for the interaction with the user and a context of the topic determined from the generated profile and the recent data; transmitting the prompt to an AI engine trained to generate a conversational output relevant to the user; receiving, through an API of the AI engine, output data specifying one or more instructions for interacting with the user; and establishing, through a communication channel with a user device of the user, a communication with the user in accordance with the one or more instructions.
Example 12. The computer-implemented system of the previous example, wherein the topic is a first topic and wherein the method further comprises: receiving, through the communication channel, input data from the user device of the user; generating of transcript of the communication based on the received input data; identifying, based on the generated transcript, one or more attributes of the user; responsive to the identifying, updating a profile of the user based on the one or more attributes; and transmitting a second topic to the AI engine, with the second topic specifying a request for another interaction with the user and further specifying the updated profile.
Example 13. The computer-implemented system of any of the previous examples, wherein the recent data associated with the user is retrieved using a digital identifier by accessing a computing system associated with the digital identifier, the computing system hosting the one or more data records through an application interface that is different from another application interface associated with one or more other data records associated with a different computing system associated with another digital identifier.
Example 14. The computer-implemented system of any of the previous examples, wherein retrieving the recent data associated with the user associated with the digital identifier comprises accessing authorization data associated with the digital identifier; and accessing, using the authorization data, a computing system hosting the one or more data records.
Example 15. The computer-implemented system of any of the previous examples, wherein retrieving the recent data associated with the user associated with the digital identifier comprises activating a sensor for initiating data collection in real time.
Example 16. The computer-implemented system of any of the previous examples, wherein the operations further comprise activating a device for generating an alert associated with the one or more instructions.
Example 17. The computer-implemented system of any of the previous examples, wherein the device comprises a medical assistance device and the alert associated with the one or more instructions corresponds to an event task.
Example 18. The computer-implemented system of any of the previous examples, wherein the operations further comprise wherein the operations further comprise determining a context for the topic, wherein the context defines behavioral and user data associated to the topic.
Example 19. The computer-implemented system of any of the previous examples, wherein the operations further comprise establishing, through an additional communication channel a communication between the user device of the user and a provider gateway system for rendering a visual representation of a service provider within a graphical user interface of the user device.
Example 20. A non-transitory, computer-readable medium storing one or more instructions executable by a computer system to perform one or more operations, comprising: simulating, by a computer system, an artificial intelligence (AI)-based digital companion for a user, comprising: onboarding the user by receiving, from a user device, one or more answers to one or more interview questions; based on the one or more received answers, generating a profile for the user; retrieving, from one or more external data sources by one or more application program interfaces (APIs), recent data associated with the user, with the recent data having occurred within a threshold amount of time; accessing, from a hardware storage device, an agenda for the user, the agenda comprising a plurality of topics to be addressed with the user; based on the generated profile, the recent data and the agenda, selecting a topic for an interaction with the user; generating a prompt, based on the topic for the interaction with the user and a context of the topic determined from the generated profile and the recent data; transmitting the prompt to an AI engine trained to generate a conversational output relevant to the user; receiving, through an API of the AI engine, output data specifying one or more instructions for interacting with the user; and establishing, through a communication channel with a user device of the user, a communication with the user in accordance with the one or more instructions.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs, that is, one or more modules of computer program instructions encoded on a tangible, non-transitory, computer-readable medium for execution by, or to control the operation of, a computer or computer-implemented system. Alternatively, or additionally, the program instructions can be encoded in/on an artificially generated propagated signal, for example, a machine-generated electrical, optical, or electromagnetic signal that is generated to encode information for transmission to a receiver apparatus for execution by a computer or computer-implemented system. The computer-storage medium can be a machine-readable storage device, a machine-readable storage substrate, a random or serial access memory device, or a combination of computer-storage mediums. Configuring one or more computers means that the one or more computers have installed hardware, firmware, or software (or combinations of hardware, firmware, and software) so that when the software is executed by the one or more computers, particular computing operations are performed. The computer storage medium is not, however, a propagated signal.

The term “real-time,” “real time,” “realtime,” “real (fast) time (RFT),” “near(ly) real-time (NRT),” “quasi real-time,” or similar terms (as understood by one of ordinary skill in the art), means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second(s), or less than 5 s. While the requested data need not be displayed (or initiated for display) instantaneously, it is displayed (or initiated for display) without any intentional delay, considering processing limitations of a described computing system and time required to, for example, gather, accurately measure, analyze, process, store, or transmit the data.

The terms “data processing apparatus,” “computer,” “computing device,” or “electronic computer device” (or an equivalent term as understood by one of ordinary skill in the art) refer to data processing hardware and encompass all kinds of apparatuses, devices, and machines for processing data, including by way of example, a programmable processor, a computer, or multiple processors or computers. The computer can also be, or further include special-purpose logic circuitry, for example, a central processing unit (CPU), a field-programmable gate array (FPGA), or an application-specific integrated circuit (ASIC). In some implementations, the computer or computer-implemented system or special-purpose logic circuitry (or a combination of the computer or computer-implemented system and special-purpose logic circuitry) can be hardware- or software-based (or a combination of both hardware-and software-based). The computer can optionally include code that creates an execution environment for computer programs, for example, code that constitutes processor firmware, a protocol stack, a database management system, an operating system, or a combination of execution environments. The present disclosure contemplates the use of a computer or computer-implemented system with an operating system, for example LINUX, UNIX, WINDOWS, MAC OS, ANDROID, or IOS, or a combination of operating systems.

A computer program, which can also be referred to or described as a program, software, a software application, a unit, a module, a software module, a script, code, or other component can be written in any form of programming language, including compiled or interpreted languages, or declarative or procedural languages, and it can be deployed in any form, including, for example, as a stand-alone program, module, component, or subroutine, for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a communication network.

While portions of the programs illustrated in the various figures can be illustrated as individual components, such as units or modules, that implement described features and functionality using various objects, methods, or other processes, the programs can instead include a number of sub-units, sub-modules, third-party services, components, libraries, and other components, as appropriate. Conversely, the features and functionality of various components can be combined into single components, as appropriate. Thresholds used to make computational determinations can be statically, dynamically, or both statically and dynamically determined.

Described methods, processes, or logic flows represent one or more examples of functionality consistent with the present disclosure and are not intended to limit the disclosure to the described or illustrated implementations, but to be accorded the widest scope consistent with described principles and features. The described methods, processes, or logic flows can be performed by one or more programmable computers executing one or more computer programs to perform functions by operating on input data and generating output data. The methods, processes, or logic flows can also be performed by, and computers can also be implemented as, special-purpose logic circuitry, for example, a CPU, an FPGA, or an ASIC.

Computers for the execution of a computer program can be based on general or special-purpose microprocessors, both, or another type of CPU. Generally, a CPU will receive instructions and data from and write to a memory. The essential elements of a computer are a CPU, for performing or executing instructions, and one or more memory devices for storing instructions and data. Generally, a computer will also include, or be operatively coupled to, receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a global positioning system (GPS) receiver, or a portable memory storage device, for example, a universal serial bus (USB) flash drive, to name just a few.

Non-transitory computer-readable media for storing computer program instructions and data can include all forms of permanent/non-permanent or volatile/non-volatile memory, media and memory devices, including by way of example semiconductor memory devices, for example, random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices; magnetic devices, for example, tape, cartridges, cassettes, internal/removable disks; magneto-optical disks; and optical memory devices, for example, digital versatile/video disc (DVD), compact disc (CD)-ROM, DVD+/−R, DVD-RAM, DVD-ROM, high-definition/density (HD)-DVD, and BLU-RAY/BLU-RAY DISC (BD), and other optical memory technologies. The memory can store various objects or data, including caches, classes, frameworks, applications, modules, backup data, jobs, web pages, web page templates, data structures, database tables, repositories storing dynamic information, or other appropriate information including any parameters, variables, algorithms, instructions, rules, constraints, or references. Additionally, the memory can include other appropriate data, such as logs, policies, security or access data, or reporting files. The processor and the memory can be supplemented by, or incorporated in, special-purpose logic circuitry.

To provide for interaction with a user, implementations of the subject matter described in this specification can be implemented on a computer having a display device, for example, a cathode ray tube (CRT), liquid crystal display (LCD), light emitting diode (LED), or plasma monitor, for displaying information to the user and a keyboard and a pointing device, for example, a mouse, trackball, or trackpad by which the user can provide input to the computer. Input can also be provided to the computer using a touchscreen, such as a tablet computer surface with pressure sensitivity or a multi-touch screen using capacitive or electric sensing. Other types of devices can be used to interact with the user. For example, feedback provided to the user can be any form of sensory feedback (such as, visual, auditory, tactile, or a combination of feedback types). Input from the user can be received in any form, including acoustic, speech, or tactile input. In addition, a computer can interact with the user by sending documents to and receiving documents from a user computing device that is used by the user (for example, by sending web pages to a web browser on a user's mobile computing device in response to requests received from the web browser).

The term “graphical user interface (GUI) can be used in the singular or the plural to describe one or more graphical user interfaces and each of the displays of a particular graphical user interface. Therefore, a GUI can represent any graphical user interface, including but not limited to, a web browser, a touch screen, or a command line interface (CLI) that processes information and efficiently presents the information results to the user. In general, a GUI can include a number of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server, or that includes a front-end component, for example, a user computer having a graphical user interface or a Web browser through which a user can interact with an implementation of the subject matter described in this specification, or any combination of one or more such back-end, middleware, or front-end components. The components of the system can be interconnected by any form or medium of wireline or wireless digital data communication (or a combination of data communication), for example, a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) using, for example, 802.11x or other protocols, all or a portion of the Internet, another communication network, or a combination of communication networks. The communication network can communicate with, for example, Internet Protocol (IP) packets, frame relay frames, Asynchronous Transfer Mode (ATM) cells, voice, video, data, or other information between network nodes.

The computing system can include users and servers. A user and server are generally remote from each other and typically interact through a communication network. The relationship of user and server arises by virtue of computer programs running on the respective computers and having a user-server relationship to each other.

While this specification contains many specific implementation details, these should not be construed as limitations on the scope of any inventive concept or on the scope of what can be claimed, but rather as descriptions of features that can be specific to particular implementations of particular inventive concepts. Certain features that are described in this specification in the context of separate implementations can also be implemented, in combination, in a single implementation. Conversely, various features that are described in the context of a single implementation can also be implemented in multiple implementations, separately, or in any sub-combination. Moreover, although previously described features can be described as acting in certain combinations and even initially claimed as such, one or more features from a claimed combination can, in some cases, be excised from the combination, and the claimed combination can be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations can be considered optional), to achieve desirable results. In certain circumstances, multitasking or parallel processing (or a combination of multitasking and parallel processing) can be advantageous and performed as deemed appropriate.

The separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the scope of the present disclosure.

Furthermore, any claimed implementation may be applicable to a computer-implemented method; a non-transitory, computer-readable medium storing computer-readable instructions to perform the computer-implemented method; and/or a computer system comprising a computer memory interoperably coupled with a hardware processor configured to perform the computer-implemented method or the instructions stored on the non-transitory, computer-readable medium.

What is claimed is:

1. A computer-implemented method comprising:
simulating, by a computer system, an artificial intelligence (AI)-based digital companion for a user, comprising:
onboarding the user by receiving, from a user device, one or more answers to one or more interview questions;
based on the one or more received answers, generating a profile for the user;
retrieving, from one or more external data sources by one or more application program interfaces (APIs), recent data associated with the user, with the recent data having occurred within a threshold amount of time;
accessing, from a hardware storage device, an agenda for the user, the agenda comprising a plurality of topics to be addressed with the user;
based on the generated profile, the recent data and the agenda, selecting a topic for an interaction with the user;
generating a prompt, based on the topic for the interaction with the user and a context of the topic determined from the generated profile and the recent data;
transmitting the prompt to an AI engine trained to generate a conversational output relevant to the user;
receiving, through an API of the AI engine, output data specifying one or more instructions for interacting with the user; and
establishing, through a communication channel with a user device of the user, a communication with the user in accordance with the one or more instructions.

2. The computer-implemented method of claim 1, wherein the topic is a first topic and wherein the method further comprises:
receiving, through the communication channel, input data from the user device of the user;
generating of transcript of the communication based on the received input data;
identifying, based on the generated transcript, one or more attributes of the user;
responsive to the identifying, updating a profile of the user based on the one or more attributes; and
transmitting a second topic to the AI engine, with the second topic specifying a request for another interaction with the user and further specifying the updated profile.

3. The computer-implemented method of claim 1, wherein the recent data associated with the user is retrieved using a digital identifier by accessing a computing system associated with the digital identifier, the computing system hosting the one or more data records through an application interface that is different from another application interface associated with one or more other data records associated with a different computing system associated with another digital identifier.

4. The computer-implemented method of claim 3, wherein retrieving the recent data associated with the user associated with the digital identifier comprises accessing authorization data associated with the digital identifier; and accessing, using the authorization data, a computing system hosting the one or more data records.

5. The computer-implemented method of claim 3, wherein retrieving the recent data associated with the user associated with the digital identifier comprises activating a sensor for initiating data collection in real time.

6. The computer-implemented method of claim 1, wherein the recent data associated with the user is constrained to the threshold amount of time relative to a current time, the threshold amount of time defining a topic currency.

7. The computer-implemented method of claim 1, comprising activating a device for generating an alert associated with the one or more instructions.

8. The computer-implemented method of claim 7, wherein the device comprises a medical assistance device and the alert associated with the one or more instructions corresponds to an event task.

9. The computer-implemented method of claim 1, comprising determining a context for the topic, wherein the context defines behavioral and user data associated to the topic.

10. The computer-implemented method of claim 1, comprising establishing, through an additional communication channel a communication between the user device of the user and a provider gateway system for rendering a visual representation of a service provider within a graphical user interface of the user device.

11. A computer-implemented system, comprising:
one or more computers; and
one or more computer memory devices interoperably coupled with the one or more computers and having tangible, non-transitory, machine-readable media storing one or more instructions that, when executed by the one or more computers, perform one or more operations, comprising:
simulating, by a computer system, an artificial intelligence (AI)-based digital companion for a user, comprising:
onboarding the user by receiving, from a user device, one or more answers to one or more interview questions;
based on the one or more received answers, generating a profile for the user;
retrieving, from one or more external data sources by one or more application program interfaces (APIs), recent data associated with the user, with the recent data having occurred within a threshold amount of time;
accessing, from a hardware storage device, an agenda for the user, the agenda comprising a plurality of topics to be addressed with the user;
based on the generated profile, the recent data and the agenda, selecting a topic for an interaction with the user;
generating a prompt, based on the topic for the interaction with the user and a context of the topic determined from the generated profile and the recent data;
transmitting the prompt to an AI engine trained to generate a conversational output relevant to the user;
receiving, through an API of the AI engine, output data specifying one or more instructions for interacting with the user; and
establishing, through a communication channel with a user device of the user, a communication with the user in accordance with the one or more instructions.

12. The computer-implemented system of claim 11, wherein the topic is a first topic and wherein the method further comprises:
receiving, through the communication channel, input data from the user device of the user;
generating of transcript of the communication based on the received input data;
identifying, based on the generated transcript, one or more attributes of the user;
responsive to the identifying, updating a profile of the user based on the one or more attributes; and
transmitting a second topic to the AI engine, with the second topic specifying a request for another interaction with the user and further specifying the updated profile.

13. The computer-implemented system of claim 11, wherein the recent data associated with the user is retrieved using a digital identifier by accessing a computing system associated with the digital identifier, the computing system hosting the one or more data records through an application interface that is different from another application interface associated with one or more other data records associated with a different computing system associated with another digital identifier.

14. The computer-implemented system of claim 13, wherein retrieving the recent data associated with the user associated with the digital identifier comprises accessing authorization data associated with the digital identifier; and accessing, using the authorization data, a computing system hosting the one or more data records.

15. The computer-implemented system of claim 13, wherein retrieving the recent data associated with the user associated with the digital identifier comprises activating a sensor for initiating data collection in real time.

16. The computer-implemented system of claim 11, wherein the operations further comprise activating a device for generating an alert associated with the one or more instructions.

17. The computer-implemented system of claim 16, wherein the device comprises a medical assistance device and the alert associated with the one or more instructions corresponds to an event task.

18. The computer-implemented system of claim 11, wherein the operations further comprise wherein the operations further comprise determining a context for the topic, wherein the context defines behavioral and user data associated to the topic.

19. The computer-implemented system of claim 11, wherein the operations further comprise establishing, through an additional communication channel a communication between the user device of the user and a provider gateway system for rendering a visual representation of a service provider within a graphical user interface of the user device.

20. A non-transitory, computer-readable medium storing one or more instructions executable by a computer system to perform one or more operations, comprising:
simulating, by a computer system, an artificial intelligence (AI)-based digital companion for a user, comprising:
onboarding the user by receiving, from a user device, one or more answers to one or more interview questions;
based on the one or more received answers, generating a profile for the user;
retrieving, from one or more external data sources by one or more application program interfaces (APIs), recent data associated with the user, with the recent data having occurred within a threshold amount of time;
accessing, from a hardware storage device, an agenda for the user, the agenda comprising a plurality of topics to be addressed with the user;
based on the generated profile, the recent data and the agenda, selecting a topic for an interaction with the user;
generating a prompt, based on the topic for the interaction with the user and a context of the topic determined from the generated profile and the recent data;
transmitting the prompt to an AI engine trained to generate a conversational output relevant to the user;
receiving, through an API of the AI engine, output data specifying one or more instructions for interacting with the user; and
establishing, through a communication channel with a user device of the user, a communication with the user in accordance with the one or more instructions.
